(19) United States
(12) Patent Application Publication
(10) Pub. No.: US 2023/0303690 A1
(43) Pub. Date: Sep. 28, 2023

(54) NOVEL ANTI-LAG3 ANTIBODIES AND METHODS OF MAKING AND USING THE SAME

(71) Applicant: SHANGHAI BENEMAE PHARMACEUTICAL CORPORATION, Shanghai (CN)

(72) Inventors: Ling ZHAN, Shanghai (CN); Yanbin MA, Shanghai (CN); Zhiqiang DU, Shanghai (CN); Yajun ZUO, Shanghai (CN); Zhenhui XIE, Shanghai (CN)

(21) Appl. No.: 18/324,935

(22) Filed: May 26, 2023

Related U.S. Application Data
(63) Continuation of application No. PCT/CN2021/134134, filed on Nov. 29, 2021.

(51) Int. Cl. C07K 16/28

(52) U.S. Cl. CPC C07K 16/2803; C07K 2317/24; C07K 2317/569; C07K 2319/30

(57) ABSTRACT

Disclosed are single domain antibodies that specifically bind to human LAG3 or a fragment or functional domain thereof, and compositions containing the single domain antibodies. Also disclosed are methods of preparing the antibodies and use of the antibodies for treating and/or preventing one or more conditions such as lymphoma, non-small cell lung cancer (NSCLC), gastric cancer, hepatocellular carcinoma, renal cell carcinoma, bladder cancer, squamous cell carcinoma of the head and neck, melanoma, or glioblastoma.

### PRIORITY CLAIM

This application is a continuation of International Pat. Application No. PCT/CN21/134134, filed on Nov. 29, 2021, which claims the benefit of International Pat. Application No. PCT/CN2020/132111, filed Nov. 27, 2020, which is incorporated by reference in its entirety, including drawings.

### SEQUENCE LISTING

The instant application contains a Sequence Listing which has been submitted electronically in ASCII format and is hereby incorporated by reference in its entirety. Said ASCII copy, created Nov. 21, 2021, is named Final_2021-11-29_57783-8019.WO01_ sequence listing.TXT and is 112 KB in size.

### TECHNICAL FIELD

This disclosure relates to novel anti-LAG3 antibodies, in particular, novel single domain antibodies, and therapeutic uses thereof.

### BACKGROUND

Lymphocyte-activation gene 3 (LAG3), also known as CD223, is a cell surface molecule expressed on activated T cells, NK cells, B cells, and plasmacytoid dendritic cells and has diverse biologic effects on T cell function [1, 2]. LAG3 is also an immune checkpoint point receptor thus a target for developing various cancer and autoimmune diseases [3]. Several anti-LAG3 antibodies are in clinical trial for treating various conditions, for example, lymphoma, non-small cell lung cancer (NSCLC), gastric cancer, hepatocellular carcinoma, renal cell carcinoma, bladder cancer, squamous cell carcinoma of the head and neck, melanoma, glioblastoma, etc. but not any anti-LAG3 antibody drug is currently available on the market. There is a need for an effective anti-LAG3 therapy. This disclosure satisfies the need in the art.

### SUMMARY

Provided herein in certain embodiments are antibodies, in particular, single domain antibodies (sdAbs), that specifically bind to LAG3 or fragments thereof. Also disclosed are CDRs of the sdAbs. In some embodiments, the antibodies are humanized antibodies. In some embodiments, the antibodies are recombinant antibodies. In some embodiments, the single domain antibodies are VHH antibodies.

Provided herein is a pharmaceutical composition for treating and/or preventing various conditions, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma. The pharmaceutical composition comprises one or more single domain antibodies disclosed herein. In some embodiments, the pharmaceutical composition comprises two or more single domain antibodies disclosed herein to produce a synergistic effect. For example, the two or more single domain antibodies bind to epitopes located at different locations or domains of the LAG3 protein. In some embodiments, the pharmaceutical composition further comprises one or more pharmaceutically acceptable adjuvants, carriers, excipients, preservatives, or a combination thereof.

Provided herein is a kit comprising one or more single domain antibodies disclosed herein for use in treating and/or preventing various conditions, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma. Alternatively, the kit comprises a pharmaceutical composition comprising one or more single domain antibodies disclosed herein for use in treating and/or preventing various conditions, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma. In certain embodiments, the kit further comprises instructions for use.

Provided herein is a method of treating and/or preventing various conditions, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma. The method includes administering to a subject in need thereof a therapeutically effective amount of one or more single domain antibodies disclosed herein. Alternatively, the method includes administering to a subject in need thereof a therapeutically effective amount of a pharmaceutical composition comprising one or more single domain antibodies disclosed herein. In certain embodiments, two or more single domain antibodies are administered to the subject simultaneously or sequentially. In certain embodiments, the pharmaceutical composition comprising two or more single domain antibodies.

Provided herein is a use of one or more single domain antibodies disclosed herein for formulating a medicament for treating and/or preventing various conditions, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma.

### BRIEF DESCRIPTION OF THE DRAWINGS

FIG. 1 shows the effects of various single domain antibodies such as sdAb Nos. 1, 17, 18, 20, 21, 24, 25, 27, and 73, in comparison to the control BMS antibody.

FIG. 2 shows the effects of various single domain antibodies such as sdAb Nos. 2015, 2018, 2093, 18, 25, and 27, in comparison to various combinations of sdAbs such as the combination 25 and 2015, the combination of 25 and 18, and the combination of 25 and 27.

FIG. 3 shows the effects of various sdAb fusions having different numbers of linkers: 25-27-2, 25-27-3, 25-Fc-27, 25-17-1, 25-17-2, 25-17-3, 25-2015-1, 25-2015-2, and 25-2015-3, in comparison to the control GS2-2 and BMS antibodies.

FIG. 4 shows binding to hLAG3-His protein with various embodiments of modified sdAb No. 17 (17 in hollow triangle): sdAb No. 17-H1 (solid square), sdAb No. 17-H2 (solid triangle), sdAb No. 17-H3 (solid reverse triangle), and sdAb No. 17-H4 (solid diamond), and using anti-PD-L1 antibody KN035 as a negative control (NC, cross).

FIG. 5 shows binding to hLAG3-His protein with another embodiment of modified sdAb No. 17 (17 in hollow triangle): sdAb No. 17-H5 (solid triangle), and using anti-PD-L1 antibody KN035 as a negative control (NC, cross).

FIG. 6 shows binding to hLAG3-His protein with another embodiment of modified sdAb No. 27 (27 in hollow triangle): sdAb No. 27-H1 (solid diamond) and sdAb No. 27-H2 (asterisk), and using anti-PD-L1 antibody KN035 as a negative control (NC, cross).

FIG. 7 shows binding to 293T-hLAG3 cells with various embodiments of modified sdAb No. 17 (17 in hollow triangle): sdAb No. 17-H1 (solid reverse triangle), sdAb No. 17-H2 (solid diamond), sdAb No. 17-H3 (solid circle), and sdAb No. 17-H4 (solid square), and using anti-PD-L1 antibody KN035 as a negative control (NC, cross).

FIG. 8 shows binding to 293T-hLAG3 cells with various embodiments of modified sdAb No. 27 (27 in hollow triangle): sdAb No. 27-H1 (solid reverse triangle) and sdAb No. 27-H2 (solid diamond), and using anti-PD-L1 antibody KN035 as a negative control (NC, cross) and BMS as a positive control (BMS, star).

FIG. 9 shows binding to hLAG3-His protein with various embodiments of modified sdAb No. 17 (17 in hollow triangle): sdAb No. 17-H5B1 (solid reverse triangle), sdAb No. 17-H5B2 (solid hexagon), sdAb No. 17-H5B3 (asterisk), and sdAb No. 17-H5 (solid circle), and using anti-PD-L1 antibody KN035 as a negative control (NC, cross).

FIG. 10 shows binding to hLAG3-His protein with various embodiments of modified sdAb No. 25 (25 in hollow triangle): sdAb No. 25-B5 (solid triangle), sdAb No. 25-B6 (solid reverse triangle), and sdAb No. 25-B7 (solid diamond), and using anti-PD-L1 antibody KN035 as a negative control (NC, cross).

FIG. 11 shows binding to hLAG3-His protein with various embodiments of modified sdAb No. 27 (27 in hollow triangle): sdAb No. 27-H1B1 (solid triangle), sdAb No. 27-H1B2 (solid reverse triangle), and sdAb No. 27-H1 B3 (solid diamond), and using anti-PD-L1 antibody KN035 as a negative control (NC, cross).

FIG. 12 shows binding to 293T-LAG3 cell (ELISA) with various embodiments of modified sdAb No. 25: sdAb No. 25-B5 (solid square), sdAb No. 25-B6 (solid triangle), and sdAb No. 25-B7 (solid reverse triangle), and using anti-PD-L1 antibody KN035 as a negative control (NC, cross).

FIG. 13 shows binding to 293T-LAG3 cell (FACS) with various embodiments of modified sdAb No. 25 (25, solid square) sdAb No. 25-B7 (solid diamond), and humanized modified sdAb No. 27 (27, solid triangle) sdAb No. 27-H1B3 (asterisk), and using anti-PD-L1 antibody KN035 as a negative control (NC, cross) and BMS as a positive control (BMS, star).

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

Provided herein are antibodies, in particular, single domain antibodies, that specifically bind to LAG3, e.g., human LAG3. The term “antibody” as used herein refers to an immunoglobulin molecule or an immunologically active portion thereof that specifically binds to, or is immunologically reactive with a particular antigen, for example, LAG3 or a functional domain or fragment thereof. The term “antibody,” in addition to natural antibodies, also includes genetically engineered or otherwise modified forms of immunoglobulins, such as synthetic antibodies, fully human antibodies, humanized antibodies. The antibodies disclosed herein, including those that are immunologically active portion of an immunoglobulin molecule, retain the ability to bind a term specific antigen, e.g., LAG3, or to bind a specific fragment or domain of LAG3. The term “single domain antibody” (sdAb) may be used interchangeably with “nanobody,” which lacks the light chains but contains only VHH of a conventional antibody. The VHH is the antigen binding fragment of heavy chain only of a conventional antibody. Unlike the conventional antibodies, the sdAb without Fc has a much smaller size, about 15 kDa or about 100 amino acids to about 150 amino acids long. For example, the sdAb is about 100 amino acids, about 110 amino acids, about 120 amino acids, about 130 amino acids, about 140 amino acids, or about 150 amino acids. Due to its small size, the sdAbs are much more stable and can recognize and specifically bind to epitopes that are not accessible by conventional antibodies.

The amino acid sequences of some examples of the single domain (VHH) antibodies disclosed herein as well as the CDRs are listed in Table 1 below.

TABLE 1

sdAb Amino Acid Sequences and CDRs

| sdAb No. | sdAb Sequence | CDR1 | CDR2 | CDR3 |
|---|---|---|---|---|
| 1 | QLQLVESGGGLVQAGGSLRLS CAASGFTFDDYA IGWFRQAPG KEREGVSCIRSSDGST YYADS VKGRFTISSDNAKNTVYLQMN SLKPEDTAVYYCAKDNYSDCR GPYHY WGQGTQVTVSS (SEQ ID NO: 1) | GFTFDDYA (SEQ ID NO: 20) | IRSSDGST (SEQ ID NO: 32) | AKDNYSDC RGPYHY (SEQ ID NO; 42) |
| 8 | QLQLVESGGGLVQPGGSLRLS CAASGFTLDYYA IGWFRQAPG KEREGVSCISSSDGST YYADS VKGRFTISRDNAKNTVYLQMN SLKPEDTAIYYCARDSLSGSG YGCRGPYDY WGQGTQVTVSS (SEQ ID NO: 2) | GFTLDYYA (SEQ ID NO: 21) | ISSSDGST (SEQ ID NO: 33) | ARDSLSGS GYGCRGPY DY (SEQ ID NO: 43) |
| 17 | QLQLVESGGGLVQPGGSLRLS CAASGLTLDGYA IGWFRQAP GKEREGLACISGSDGST HYLD SVKGRFTISRDNGKNTMALQM NSLQFGDTAIYYCAARRGSCS GSWTY WGQGTQVTVSS (SEQ ID NO: 3) | GLTLDGYA (SEQ ID NO: 22) | ISGSDGST (SEQ ID NO: 34) | AARRGSCS GSWTY (SEQ ID NO: 44) |
| 18 | QVQLVESGGGLVQPGGSLRL SCAASGFTLNEHA IGWFRRAP GKEREGVSCISSSDGST YYHD | GFTLNEHA (SEQ ID NO: | ISSSDGST (SEQ ID NO: | AAALGRDC QGGQWKTS S (SEQ ID |
| | SVKGRFNMSRDDAKNSVYLQ MSSLKPEDTAIYYCAAALGRD CQGGQWKTSS WGQGTQVTV SS (SEQ ID NO: 4) | 23) | 33) | NO: 45) |
| 20 | QVQLVESGGGLVQPGGSLRL SCAASGLTLDGYA VGWFRQA PGKDREGLSCISGSDGST HYL DSVKGRFTISRDNAKSTMALQ MNSLQFGDTAIYYCAARRGSC SGSWTY WGQGTQVTVSS (SEQ ID NO: 5) | GLTLDGYA (SEQ ID NO: 22) | ISGSDGST (SEQ ID NO: 34) | AARRGSCS GSWTY (SEQ ID NO: 44) |
| 21 | QLQLVESGGGLVQPGGSLRLS CAASGLTLDGYAIAWFRQAPG KEREGLSCISGTDGST HYLDS VKGRFTISRDNGKNTMALQMN SLQFGDTAIYYCAARRGSCSG SWTY WGQGTQITVSS (SEQ ID NO: 6) | GLTLDGYA (SEQ ID NO: 22) | ISGTDGST (SEQ ID NO: 35) | AARRGSCS GSWTY (SEQ ID NO: 44) |
| 24 | QLQLVESGGGLVQPGGSLRLS CAASGFTLNEHA IGWFRQAPG KEREGVSCISSSDGST YYHDS VKGRFTMSRDDAKNTVYLQM NSLKAEDTAIYYCAAALGRDC QGGQWKTSS WGQGTQVTVS S (SEQ ID NO: 7) | GFTLNEHA (SEQ ID NO: 23) | ISSSDGST (SEQ ID NO: 33) | AAALGRDC QGGQWKTS S (SEQ ID NO: 45) |
| 25 | QLQLVESGGGLVQAGGSLRLS CAASGSIESFSF MGWYRQAP GKQRELVAGFTGAASLSFS TY TDAVKGRFTISRDNAKNTVYL QMNSLKPEDTAVYFCNVDPW SQSWPGRYWGQGTQVTVSS (SEQ ID NO: 8) | GSIESFSF (SEQ ID NO: 24) | FTGAASLS FS (SEQ ID NO: 36) | NVDPWSQS WPGRY (SEQ ID NO: 46) |
| 27 | QVQLVESGGGLVQPGGSLRL SCAASGFTLDYYA IGWFRQAP GKEREGVSCIRSSDGSTYYAD SVKGRFTISSDNTKNTVYLQM NSLKPEDTAVYYCATDRYSDC RGPYEN WGQGTQVTVSS (SEQ ID NO: 9) | GFTLDYYA (SEQ ID NO: 21) | IRSSDGST (SEQ ID NO: 32) | ATDRYSDC RGPYEN (SEQ ID NO: 47) |
| 73 | QVQLVESGGGLVQPGGSLKL SCTASGFTLDYYA IAWFRQAP GKEREAVSCISSSDGST RYAD SVKGRFTISRDNAKNTVYLQM NSLKPEDTAVYYCAADPYGDC | GFTLDYYA (SEQ ID NO: 21) | ISSSDGST (SEQ ID NO: 33) | AADPYGDC RGPYDY (SEQ ID NO: 48) |
| | RGPYDY WGQGTQVTVSS (SEQ ID NO: 10) | | | |
| 2002 | QLQLVESGGGLVQAGGSLRLS CAASGFFFDDYA IAWFRQAPG KDREGVSCISSSDGST VYADS VKGRFTSSRDNAKDTVDLQM NSLKPEDTAVYYCAVRRGYG CSGPWHY WGQGTQVTVSS (SEQ ID NO: 11) | GFFFDDYA (SEQ ID NO: 25) | ISSSDGST (SEQ ID NO: 33) | AVRRGYGC SGPWHY (SEQ ID NO: 49) |
| 2012 | QVQLVESGGGLVQPGGSLRL SCAASGFTLNEHAI GWFRQAP GKEREGVSCISSSDGST YYHD SVKGRFTMSRDDAKNTVYLQ MNSLKPEDTAIYYCAAALGRD CQGGQWKTDS WGQGTQVTV SS (SEQ ID NO: 12) | GFTLNEHA (SEQ ID NO: 23) | ISSSDGST (SEQ ID NO: 33) | AAALGRDC QGGQWKTD S (SEQ ID NO: 50) |
| 2015 | QLQLVESGGGLAQTGGSLRLS CAAAGFTFDDTA TGWFRQAP GKNREGVACISSSDGRI HYIDS AKGRFTISKDNAKKTVYLQMD SLKPEDTAVYYCAAARKGSTD CQGAIWGS WGQGTQVIVSS (SEQ ID NO: 13) | GFTFDDTA (SEQ ID NO: 26) | ISSSDGRI (SEQ ID NO: 37) | AAARKGST DCQGAIWG S (SEQ ID NO: 51) |
| 2018 | QVQLVESGGGLVQPGGSLKL SCTASGFTLDYYA IAWFRQAP GKEREAVSCISSSDGST RYAD SVKGRFTISRDNAKNTVYLQM NSLKPEDTAVYYCAADPYGDC RGPYDY WGQGTQVTVSS (SEQ ID NO: 14) | GFTLDYYA (SEQ ID NO: 21) | ISSSDGST (SEQ ID NO: 33) | AADPYGDC RGPYDY (SEQ ID NO: 48) |
| 2021 | QLQLVESGGGLVQPGGSLRLS CAASGFTLDSYA IGWFRQAPG KEREAVSCITSSDGRT YYADS VKGRFTISRDNAKNTVYLQMN SLKPEDTAVYYCAADRWQTC TGPYDH WGQGTQVTVSS (SEQ ID NO: 15) | GFTLDSYA (SEQ ID NO: 27) | ITSSDGRT (SEQ ID NO: 38) | AADRWQTC TGPYDH (SEQ ID NO: 52) |
| 2054 | QVQLVESGGGLVQPGGSLRL SCAASGFTLDNYA IGWFRQAP GKEREGVSCISRNAGST KYAD SVKGRFTISRDNAKNTVYLQM NSLKPEDTAVYYCAARVPDYG DITDCGGKWDDY WGPGTQVT | GFTLDNYA (SEQ ID NO: 28) | ISRNAGST (SEQ ID NO: 39) | AARVPDYG DITDCGGK WDDY (SEQ ID NO: 53) |
| | VSS (SEQ ID NO: 16) | | | |
| 2057 | QLQLVESGGGLVQTGGSLRLS CAASGFIFDDYS IGWFRQAPG RHREVVSCISSSDTKY ADSAK GRFTISRNNAEDTVYLQMNSL KPEDTAVYYCAARSGDGCNG RWDY WGQGTQVTVSS (SEQ ID NO: 17) | GFIFDDYS (SEQ ID NO: 29) | ISSSDTKY (SEQ ID NO; 40) | AARSGDGC NGRWDY (SEQ ID NO: 54) |
| 2093 | QVQLVESGGGLVQPGGSLTLS CVASGFTLDYYT VGWFRQVP GKEREGISCISSSDGST RYAD SVKGRFTISRDNAKNTMSLQM NSLKPEDTAVYYCATPGIHGT SCNTSWKY YGQGTQVTVSS (SEQ ID NO: 18) | GFTLDYYT (SEQ ID NO: 30) | ISSSDGST (SEQ ID NO: 33) | ATPGIHGTS CNTSWKY (SEQ ID NO: 55) |
| 2095 | QVQLVESGGGLVQPGGSLRLSCVASGFTLDDYA LGWFRQA PGKEREAVSCISGSDGGT HYH DSVKGRFTISKNKAKNTLYLQ MNSLKPEDTAVYYCARAQAD RCLGGIWPTGD WGQGTQVTV SS (SEQ ID NO: 19) | GFTLDDYA (SEQ ID NO: 31) | ISGSDGGT (SEQ ID NO: 41) | ARAQADRC LGGIWPTGD (SEQ ID NO: 56) |

Note: The CDR regions were calculated by the IMGT numbering methods.

In some embodiments, disclosed are sdAb fusions obtained by linking two sdAbs with one or more G4S (GGGGS) (SEQ ID NO: 57) linkers. For example, one, two, three, four, five, or six G4S linkers can be used to link two sdAbs, thereby to obtain an sdAb fusion. The amino acid sequences of some examples of the sdAb fusions disclosed herein are listed in Table 2 below. One or more G4S linkers (SEQ ID NO: 57) are highlighted, as well as the signal peptide at the N-terminus and the partial Fc sequence at the C-terminus.

TABLE 2

sdAb Fusion Sequences

| | | |
|---|---|---|
| sdAb Conjugate | Description | Amino Acid Sequence |
| 25-27-1 | C-terminus of sdAb No. 25 fused to N-terminus of sdAb No. 27 by one G4S | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSQV QLVESGGGLVQPGGSLRLSCAASGFTLDYYAIGWFRQ APGKEREGVSCIRSSDGSTYYADSVKGRFTISSDNTKN |
| | linker | TVYLQMNSLKPEDTAVYYCATDRYSDCRGPYENWGQ GTQVTVSSEPKSCDKTHTCPPCPAPELLG (SEQ ID NO: 58) |
| 27-25-1 | C-terminus of sdAb No. 27 fused to N-terminus of sdAb No. 25 by one G4S linker | MGWSCIILFLVATATGVHSQVQLVESGGGLVQPGGSL RLSCAASGFTLDYYAIGWFRQAPGKEREGVSCIRSSDG STYYADSVKGRFTISSDNTKNTVYLQMNSLKPEDTAVY YCATDRYSDCRGPYENWGQGTQVTVSSGGGGSQLQL VESGGGLVQAGGSLRLSCAASGSIESFSFMGWYRQAP GKQRELVAGFTGAASLSFSTYTDAVKGRFTISRDNAKN TVYLQMNSLKPEDTAVYFCNVDPWSQSWPGRYWGQG TQVTVSSEPKSCDKTHTCPPCPAPELLG (SEQ ID NO: 59) |
| 25-27-2 | C-terminus of sdAb No. 25 fused to N-terminus of sdAb No. 27 by two G4S linkers | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGGGGSQVQLVESGGGLVQPGGSLRLSCAASGFTLDYYAI GWFRQAPGKEREGVSCIRSSDGSTYYADSVKGRFTISS DNTKNTVYLQMNSLKPEDTAVYYCATDRYSDCRGPYE NWGQGTQVTVSSEPKSCDKTHTCPPCPAPELLG (SEQ ID NO: 60) |
| 27-25-2 | C-terminus of sdAb No. 27 fused to N-terminus of sdAb No. 25 by two G4S linkers | MGWSCIILFLVATATGVHSQVQLVESGGGLVQPGGSL RLSCAASGFTLDYYAIGWFRQAPGKEREGVSCIRSSDG STYYADSVKGRFTISSDNTKNTVYLQMNSLKPEDTAVY YCATDRYSDCRGPYENWGQGTQVTVSSGGGGSGGG GSQLQLVESGGGLVQAGGSLRLSCAASGSIESFSFMG WYRQAPGKQRELVAGFTGAASLSFSTYTDAVKGRFTIS RDNAKNTVYLQMNSLKPEDTAVYFCNVDPWSQSWPG RYWGQGTQVTVSSEPKSCDKTHTCPPCPAPELLG (SEQ ID NO: 61) |
| 25-27-3 | C-terminus of sdAb No. 25 fused to N-terminus of sdAb No. 27 by three G4S linkers | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGG GGSGGGGSQVQLVESGGGLVQPGGSLRLSCAASGFT LDYYAIGWFRQAPGKEREGVSCIRSSDGSTYYADSVKG RFTISSDNTKNTVYLQMNSLKPEDTAVYYCATDRYSDC RGPYENWGQGTQVTVSSEPKSCDKTHTCPPCPAPEL LG (SEQ ID NO: 62) |
| 27-25-3 | C-terminus of sdAb No. 27 fused to N-terminus of sdAb No. 25 by three G4S | MGWSCIILFLVATATGVHSQVQLVESGGGLVQPGGSL RLSCAASGFTLDYYAIGWFRQAPGKEREGVSCIRSSDG STYYADSVKGRFTISSDNTKNTVYLQMNSLKPEDTAVY YCATDRYSDCRGPYENWGQGTQVTVSSGGGGSGGG GSGGGGSQLQLVESGGGLVQAGGSLRLSCAASGSIES FSFMGWYRQAPGKQRELVAGFTGAASLSFSTYTDAVK |
| | linkers | GRFTISRDNAKNTVYLQMNSLKPEDTAVYFCNVDPWS QSWPGRYWGQGTQVTVSSEPKSCDKTHTCPPCPAPE LLG (SEQ ID NO: 63) |
| 25-17-1 | C-terminus of sdAb No. 25 fused to N-terminus of sdAb No. 17 by one G4S linker | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSQL QLVESGGGLVQPGGSLRLSCAASGLTLDGYAIGWFRQ APGKEREGLACISGSDGSTHYLDSVKGRFTISRDNGKN TMALQMNSLQFGDTAIYYCAARRGSCSGSWTYWGQG TQVTVSS (SEQ ID NO: 64) |
| 17-25-1 | C-terminus of sdAb No. 17 fused to N-terminus of sdAb No. 25 by one G4S linker | MGWSCIILFLVATATGVHSQLQLVESGGGLVQPGGSLR LSCAASGLTLDGYAIGWFRQAPGKEREGLACISGSDGS THYLDSVKGRFTISRDNGKNTMALQMNSLQFGDTAIYY CAARRGSCSGSWTYWGQGTQVTVSSGGGGSQLQLV ESGGGLVQAGGSLRLSCAASGSIESFSFMGWYRQAPG KQRELVAGFTGAASLSFSTYTDAVKGRFTISRDNAKNT VYLQMNSLKPEDTAVYFCNVDPWSQSWPGRYWGQGT QVTVSSEPKSCDKTHTCPPCPAPELLG (SEQ ID NO: 65) |
| 25-17-2 | C-terminus of sdAb No. 25 fused to N-terminus of sdAb No. 17 by two G4S linkers | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGG GGSQLQLVESGGGLVQPGGSLRLSCAASGLTLDGYAI GWFRQAPGKEREGLACISGSDGSTHYLDSVKGRFTISR DNGKNTMALQMNSLQFGDTAIYYCAARRGSCSGSWTY WGQGTQVTVSSEPKSCDKTHTCPPCPAPELLG (SEQ ID NO: 66) |
| 17-25-2 | C-terminus of sdAb No. 17 fused to N-terminus of sdAb No. 25 by two G4S linkers | MGWSCIILFLVATATGVHSQLQLVESGGGLVQPGGSLR LSCAASGLTLDGYAIGWFRQAPGKEREGLACISGSDGS THYLDSVKGRFTISRDNGKNTMALQMNSLQFGDTAIYY CAARRGSCSGSWTYWGQGTQVTVSSGGGGSGGGGS QLQLVESGGGLVQAGGSLRLSCAASGSIESFSFMGWY RQAPGKQRELVAGFTGAASLSFSTYTDAVKGRFTISRD NAKNTVYLQMNSLKPEDTAVYFCNVDPWSQSWPGRY WGQGTQVTVSSEPKSCDKTHTCPPCPAPELLG (SEQ ID NO: 67) |
| 25-17-3 | C-terminus of sdAb No. 25 fused to N-terminus of sdAb No. 17 by three G4S | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGG GGSGGGGSQLQLVESGGGLVQPGGSLRLSCAASGLTL DGYAIGWFRQAPGKEREGLACISGSDGSTHYLDSVKG RFTISRDNGKNTMALQMNSLQFGDTAIYYCAARRGSCS |
| | linkers | GSWTYWGQGTQVTVSSEPKSCDKTHTCPPCPAPELL G (SEQ ID NO: 68) |
| 17-25-3 | C-terminus of sdAb No. 17 fused to N-terminus of sdAb No. 25 by three G4S linkers | MGWSCIILFLVATATGVHSQLQLVESGGGLVQPGGSLR LSCAASGLTLDGYAIGWFRQAPGKEREGLACISGSDGS THYLDSVKGRFTISRDNGKNTMALQMNSLQFGDTAIYY CAARRGSCSGSWTYWGQGTQVTVSSGGGGSGGGGS GGGGSQLQLVESGGGLVQAGGSLRLSCAASGSIESFS FMGWYRQAPGKQRELVAGFTGAASLSFSTYTDAVKGR FTISRDNAKNTVYLQMNSLKPEDTAVYFCNVDPWSQS WPGRYWGQGTQVTVSSEPKSCDKTHTCPPCPAPELL G (SEQ ID NO: 69) |
| 25-2015-1 | C-terminus of sdAb No. 25 fused to N-terminus of sdAb No. 2015 by one G4S linker | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSQL QLVESGGGLAQTGGSLRLSCAAAGFTFDDTATGWFRQ APGKNREGVACISSSDGRIHYIDSAKGRFTISKDNAKKT VYLQMDSLKPEDTAVYYCAAARKGSTDCQGAIWGSWG QGTQVIVSSEPKSCDKTHTCPPCPAPELLG (SEQ ID NO: 70) |
| 2015-25-1 | C-terminus of sdAb No. 2015 fused to N-terminus of sdAb No. 25 by one G4S linker | MGWSCIILFLVATATGVHSQLQLVESGGGLAQTGGSLR LSCAAAGFTFDDTATGWFRQAPGKNREGVACISSSDG RIHYIDSAKGRFTISKDNAKKTVYLQMDSLKPEDTAVYY CAAARKGSTDCQGAIWGSWGQGTQVIVSSGGGGSQL QLVESGGGLVQAGGSLRLSCAASGSIESFSFMGWYRQ APGKQRELVAGFTGAASLSFSTYTDAVKGRFTISRDNA KNTVYLQMNSLKPEDTAVYFCNVDPWSQSWPGRYWG QGTQVTVSSEPKSCDKTHTCPPCPAPELLG (SEQ ID NO: 71) |
| 25-2015-2 | C-terminus of sdAb No. 25 fused to N-terminus of sdAb No. 2015 by two G4S linkers | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGGGGSQLQLVESGGGLAQTGGSLRLSCAAAGFTFDDTAT GWFRQAPGKNREGVACISSSDGRIHYIDSAKGRFTISK DNAKKTVYLQMDSLKPEDTAVYYCAAARKGSTDCQGAI WGSWGQGTQVIVSSEPKSCDKTHTCPPCPAPELLG (SEQ ID NO: 72) |
| 2015-25-2 | C-terminus of sdAb No. 2015 fused to N-terminus of sdAb No. 25 by two G4S linkers | MGWSCIILFLVATATGVHSQLQLVESGGGLAQTGGSLR LSCAAAGFTFDDTATGWFRQAPGKNREGVACISSSDG RIHYIDSAKGRFTISKDNAKKTVYLQMDSLKPEDTAVYY CAAARKGSTDCQGAIWGSWGQGTQVIVSSGGGGSGG GGSQLQLVESGGGLVQAGGSLRLSCAASGSIESFSFM GWYRQAPGKQRELVAGFTGAASLSFSTYTDAVKGRFTI SRDNAKNTVYLQMNSLKPEDTAVYFCNVDPWSQSWP GRYWGQGTQVTVSSEPKSCDKTHTCPPCPAPELLG (SEQ ID NO: 73) |
| 25-2015-3 | C-terminus of sdAb No. 25 fused to N-terminus of sdAb No. 2015 by three G4S linkers | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSSGGGGSGG GGSGGGGSQLQLVESGGGLAQTGGSLRLSCAAAGFTF DDTATGWFRQAPGKNREGVACISSSDGRIHYIDSAKGR FTISKDNAKKTVYLQMDSLKPEDTAVYYCAAARKGSTD CQGAIWGSWGQGTQVIVSSEPKSCDKTHTCPPCPAPE LLG (SEQ ID NO: 74) |
| 2015-25-3 | C-terminus of sdAb No. 2015 fused to N-terminus of sdAb No. 25 by three G4S linkers | MGWSCIILFLVATATGVHSQLQLVESGGGLAQTGGSLR LSCAAAGFTFDDTATGWFRQAPGKNREGVACISSSDG RIHYIDSAKGRFTISKDNAKKTVYLQMDSLKPEDTAVYY CAAARKGSTDCQGAIWGSWGQGTQVIVSSGGGGSGG GGSGGGGSQLQLVESGGGLVQAGGSLRLSCAASGSIE SFSFMGWYRQAPGKQRELVAGFTGAASLSFSTYTDAV KGRFTISRDNAKNTVYLQMNSLKPEDTAVYFCNVDPWS QSWPGRYWGQGTQVTVSSEPKSCDKTHTCPPCPAPE LLG (SEQ ID NO: 75) |
| 25-Fc-27 | C-terminus of sdAb No. 25 fused to an Fc fragment, and then to N-terminus of sdAb No. 27 by one G4S linker * Fc fragment is shown in parentheses. | MGWSCIILFLVATATGVHSQLQLVESGGGLVQAGGSLR LSCAASGSIESFSFMGWYRQAPGKQRELVAGFTGAAS LSFSTYTDAVKGRFTISRDNAKNTVYLQMNSLKPEDTA VYFCNVDPWSQSWPGRYWGQGTQVTVSS(EPKSCDK THTCPPCPAPELLGGPSVFLFPPKPKDTLMISRTPEVTC VVVDVSHEDPEVKFNWYVDGVEVHNAKTKPREEQYAS TYRVVSVLTVLHQDWLNGKEYKCKVSNKALPAPIEKTIS KAKGQPREPQVYTLPPSREEMTKNQVSLTCLVKGFYP SDIAVEWESNGQPENNYKTTPPVLDSDGSFFLYSKLTV DKSRWQQGNVFSCSVMHEALHNHYTQKSLSLSPGK)G GGGSQVQLVESGGGLVQPGGSLRLSCAASGFTLDYYA IGWFRQAPGKEREGVSCIRSSDGSTYYADSVKGRFTIS SDNTKNTVYLQMNSLKPEDTAVYYCATDRYSDCRGPY ENWGQGTQVTVSS (SEQ ID NO: 76) |

The antibodies provided herein include variants of the sequences disclosed herein that contain one or more mutations in their amino acid sequences while retaining binding affinity for LAG3 and/or a fragment or a functional domain thereof. In some embodiments, disclosed is an sdAb comprising, consisting essentially of, or consisting of an amino acid sequence that is at least 85%, at least 90%, at least 95%, at least 98%, or at least 99% identical to a sequence selected from the group consisting of SEQ ID NOs: 1-19, 80-86, and 89-97, or a fragment thereof that retains binding affinity for LAG3 and/or a fragment or a functional domain thereof. In some embodiments, disclosed herein is an sdAb comprising a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, or a CDR3 having an amino acid sequence selected from the group consisting of SEQ ID NOs: 42-56, 108, 109, and 112. In some embodiments, disclosed herein is an sdAb comprising a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, and a CDR3 having an amino acid sequence selected from the group consisting of SEQ ID NOs: 42-56, 108, 109, and 112. In certain embodiments, a variant of the sequence disclosed herein contains one or more mutations such that one or more DG of one or more of CDRs are mutated to DA and/or EG; and/or one or more mutations such that one or more NG of one or more of CDRs are mutated to NA and/or QG, wherein D is aspartic acid, G is glycine, A is alanine, E is glutamic acid, N is asparagine, and Q is glutamine.

Also disclosed is an sdAb fusion comprising two or more sdAbs, each sdAb comprising, consisting essentially of, or consisting of an amino acid sequence selected from the group consisting of SEQ ID NO: 1-19; 80-86, and 89-97, each sdAb comprising a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, or a CDR3 having an amino acid sequence selected from the group consisting of SEQ ID NOs: 42-56, 108, 109, and 112; or each sdAb comprising a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, and a CDR3 having an amino acid sequence selected from the group consisting of SEQ ID NOs: 42-56, 108, 109, and 112. One skilled in the art would understand that any two or more sdAbs disclosed herein can be combined or can be fused to form a sdAb fusion.

In some embodiments, the two or more sdAbs are fused via one or more G4S linkers. In some embodiments, the sdAb fusion further comprises an Fc region or a fragment thereof. In some embodiments, disclosed herein is an sdAb fusion comprising, consisting of, or consisting essentially of the amino acid sequence selected from the group consisting of SEQ ID NOs: 58-76, or an amino acid sequence that is at least 85%, at least 90%, at least 95%, at least 98%, or at least 99% identical to a sequence selected from the group consisting of SEQ ID NOs: 58-76.

### Pharmaceutical Compositions

One or more anti-LAG3 antibodies or fusions disclosed herein can be formulated into pharmaceutical compositions. The pharmaceutical compositions may further comprise one or more pharmaceutically acceptable carriers, excipients, preservatives, or a combination thereof. The pharmaceutical compositions can have various formulations, e.g., injectable formulations, lyophilized formulations, liquid formulations, etc. Depending on the formulation and administration route, one would select suitable additives, such as adjuvants, carriers, excipients, preservatives. See, for example, Wang et al., J. Pharm. Sciences 96(1): 1-26 (2007), the content of which is incorporated by reference.

In certain embodiments, the pharmaceutical composition may further comprise one or more additional antibodies such as an anti-PD-1 antibody, an anti-PD-L1 antibody, a CTLA-4 antibody, or a combination thereof. The one or more additional antibodies may be formulated into the same pharmaceutical composition comprising the anti-LAG3 antibody disclosed herein or into separate pharmaceutical compositions for combinational therapy.

The pharmaceutical composition can be included in a kit with an instruction for using the composition.

### Methods of Treatment

Provided herein is a method of treating and/or preventing cancer or an autoimmune disease in a subject suffering from and/or at an elevated risk of developing the cancer or autoimmune disease. The diseases include, for example, lymphoma, non-small cell lung cancer (NSCLC), gastric cancer, hepatocellular carcinoma, renal cell carcinoma, bladder cancer, squamous cell carcinoma of the head and neck, melanoma, and glioblastoma. The method entails administering a therapeutically effective amount of an anti-LAG3 antibody provided herein to the subject. In some embodiments, the method comprises administering a pharmaceutical composition comprising an anti-LAG3 antibody as provided herein to the subject. One or more additional antibodies such as anti-PD-1 antibodies and/or anti-PD-L1 antibodies also can be administered in combination with the anti-LAG3 antibody disclosed herein.

As used herein, the term “subject” refers to a mammalian subject, preferably a human. A “subject in need thereof” refers to a subject who has been diagnosed with cancer or an autoimmune disease, or is at an elevated risk of developing cancer or an autoimmune disease. The phrases “subject” and “patient” are used interchangeably herein.

The terms “treat,” “treating,” and “treatment” as used herein with regard to a condition refers to alleviating the condition partially or entirely, preventing the condition, decreasing the likelihood of occurrence or recurrence of the condition, slowing the progression or development of the condition, or eliminating, reducing, or slowing the development of one or more symptoms associated with the condition. With regard to cancer or an autoimmune disease, “treating” may refer to preventing or slowing the existing tumor from growing larger, preventing or slowing the formation or metastasis of cancer, and/or slowing the development of certain symptoms of the cancer or autoimmune disease. In some embodiments, the term “treat,” “treating,” or “treatment” means that the subject has a reduced number or size of tumor comparing to a subject without being administered with the antibodies. In some embodiments, the term “treat,” “treating,” or “treatment” means that one or more symptoms of the cancer or autoimmune disease are alleviated in a subject receiving an antibody or pharmaceutical composition as disclosed herein comparing to a subject who does not receive such treatment.

A “therapeutically effective amount” of an antibody or pharmaceutical composition as used herein is an amount of the antibody or pharmaceutical composition that produces a desired therapeutic effect in a subject, such as treating and/or preventing cancer or an autoimmune disease. In certain embodiments, the therapeutically effective amount is an amount of the antibody or pharmaceutical composition that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including but not limited to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject’s response to administration of the antibody or the pharmaceutical composition and adjusting the dosage accordingly. For additional guidance, see, e.g., Remington: The Science and Practice of Pharmacy, 22^{nd} Edition, Pharmaceutical Press, London, 2012, and Goodman & Gilman’s The Pharmacological Basis of Therapeutics, 12^{th} Edition, McGraw-Hill, New York, NY, 2011, the entire disclosures of which are incorporated by reference herein.

In some embodiments, a therapeutically effective amount of an antibody disclosed herein is in the range from about 0.01 mg/kg to about 30 mg/kg, from about 0.1 mg/kg to about 10 mg/kg, from about 1 mg/kg to about 5 mg/kg.

It is within the purview of one of ordinary skill in the art to select a suitable administration route, such as subcutaneous administration, intravenous administration, intramuscular administration, intradermal administration, intrathecal administration, or intraperitoneal administration. For treating a subject in need thereof, the antibody or pharmaceutical composition can be administered continuously or intermittently, for an immediate release, controlled release or sustained release. Additionally, the antibody or pharmaceutical composition can be administered three times a day, twice a day, or once a day for a period of 3 days, 5 days, 7 days, 10 days, 2 weeks, 3 weeks, or 4 weeks. The antibody or pharmaceutical composition may be administered over a pre-determined time period. Alternatively, the antibody or pharmaceutical composition may be administered until a particular therapeutic benchmark is reached. In certain embodiments, the methods provided herein include a step of evaluating one or more therapeutic benchmarks to determine whether to continue administration of the antibody or pharmaceutical composition.

The following examples are provided to better illustrate the embodiments and are not to be interpreted as limiting the scope of any claimed embodiment. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1: Generation of Anti-LAG3 Single Domain Antibodies

The following antigens were purchased from Sino Biological company: LAG3 Protein, Human, Recombinant, Biotinylated (Catalog No. 16498-HNAH-B), LAG3 Protein, Human, Recombinant (Fc Tag) (Catalog No. 16498-H02H), and LAG3 Protein, Human, Recombinant (His Tag) (Catalog No. 16498-H08H).

The alpacas, aged one to two years old from Australia, were immunized according to the following schedule:

TABLE 3

Alpaca Immunization Schedule

| Days | Action |
|---|---|
| 1 | Negative control of 10 mL peripheral blood was collected before immunization. |
| | Antigen (1 mg) and Complete Freund’s Adjuvant (CFA) (1 mL) were mixed well and injected subcutaneously. |
| 21 | Antigen (0.5 mg) and CFA (1 mL) were mixed well and injected subcutaneously. |
| 28 | 10 mL peripheral blood was collected for sera antibody detection. |
| 42 | Antigen (Fc tag) (0.5 mg) and Incomplete Freund’s Adjuvant (IFA) (1 mL) were mixed well and injected subcutaneously. |
| 49 | 50 mL peripheral blood was collected for isolating PBMCs. |
| 63 | Antigen (Fc tag) (0.5 mg) and IFA (1 mL) were mixed well and injected subcutaneously. |
| 70 | 50 mL peripheral blood was collected for isolating PBMCs. |

Microtiter plates were coated with recombinant LAG3 fusion protein at 2 µg/mL diluted in PBS, 100 µL/well incubated overnight at 4° C., then blocked with 300 µL/well 3% evaporated milk incubated at 37° C. for 1 hour. The plates were washed three times with phosphate buffered saline with Tween 20 (PBST). 100 µl of dilutions of plasma from LAG3-immunized alpaca were added to each well and incubated at 37° C. for 45 minutes. The plates were washed five times with PBST and then incubated with a goat anti-alpaca antibody conjugated with Horse Radish Peroxidase (HRP) (diluted 1:1 with PBS), 100 µL/well, for 1 hour at room temperature. After washing five times with PBST, the plates were developed with tetramethylbenzidine (TMB) substrate, 100 µL/well, and incubated at 37° C. for 5 minutes before the termination buffer was added at 50 µL/well and analyzed by spectrophotometer at OD 450 nm.

TABLE 4

Serum Titer After Fourth Immunization

| Dilution Fold | Serum from alpaca immunized with LAG-3 |
|---|---|
| 1:2000 | 2.212 |
| 1:4000 | 1.614 |
| 1:8000 | 1.241 |
| 1:16000 | 0.855 |
| 1:32000 | 0.432 |
| 1:64000 | 0.311 |
| 1:128000 | 0.214 |
| Blank | 0.064 |

### Example 2: Construction of Nanobody Immune Libraries

PBMCs were separated from 50 mL peripheral blood. Total RNA was extracted from the PBMCs by TRIzol (Invitrogen) according to the manufacturer’s instructions. From this cDNA, the single domain antibody encoding open reading frames can be amplified by PCR and cloned into an appropriate phage display vector. The VHH fragments were cloned into M13 phagemid vector containing 6×His tags. The resulting library size was 5.2×10⁸ cfu/mL (52*100*10⁵).

### Example 3: Selection by Phage Display

Affinity biopanning: 96-well plates were coated with 100 µl/well of 5 µg/mL LAG-3 protein diluted in carbonate buffer solution and incubated overnight at 4° C. The coating buffer was discarded and the plates were washed three times with PBS. 300 µL/well 3% BSA-PBS blocking buffer was added and incubated at 37° C. for one hour. The plates were washed three times with PBS and 100 µL/well of the phage library was added and incubated at 37° C. for one hour. The unbound phage was pipetted out and the plates were washed six times with PBST and two times with PBS. 100 µL elution buffer (Gly—HCl) was added to each well and incubated at 37° C. for 8 minutes. The elute containing specifically bound phage was transferred into a clean 1.5 mL microcentrifuge tube, and pH was neutralized with 15 µL Tris-HCl buffer immediately. 10 µL of the solution was taken out and subjected to 10-fold serial dilution. The phage titer was estimated by counting the colonies from the highest dilutions. The biopanning condition of each round is detailed in Table 5 below.

TABLE 5

Biopanning Conditions

| Round | Antigen coating (µg/mL) | Blocking buffer | Phage library cDNA input | Incubation time (hour) | PBST | PBST wash times |
|---|---|---|---|---|---|---|
| | | | (cfu) | | | |
| 1 | 5 | BSA-PBS | 2×10¹¹ | 1 | 0.1% | 6 |
| 2 | 2 | OVA-PBS | 1×10¹¹ | 0.75 | 0.25% | 8 |
| 3 | 1 | BSA-PBS | 1×10¹¹ | 0.5 | 0.5% | 8 |

Rescue and amplification of phage from immune libraries: The phage library elute was mixed with 20 mL TG1 E. coli cells that reached logarithmic phase, incubated at 37° C. without shaking for 30 minutes. 1 ml of 20% glucose and 4 µL of ampicillin was added to the tube and incubated at 37° C. with rotating at 180 rpm for 30 minutes. M13K07 helper phage was added at a ratio of cell: phage = 1:20 and incubated at 37° C. without shaking for 30 minutes. Then 20 mL 2x YT was added and incubated at 37° C. with rotating at 180 rpm for 30 minutes. The supernatant was transferred to a new microcentrifuge tube, centrifuged for 10 minutes at 5000 rpm. The precipitated phage was resuspended in 50 mL 2× YT with ampicillin and kanamycin and incubated overnight at 30° C. with rotating at 230 rpm. The overnight culture was centrifuged at 10,000 rpm at room temperature for 20 minutes. The supernatant was transferred to a new microcentrifuge tube, PEG/NaCl solution was added at a ratio of 1:5 (v/v), mixed gently and incubated for 1 hour at 4° C. The solution was centrifuged at 10,000 rpm at 4° C. for 20 minutes. The supernatant was discarded and the precipitate was resuspended in 1 mL PBS, and PEG/NaCl solution was added to the supernatant at a ratio of 1:5 (v/v), and incubated at 4° C. for 1 hour. The solution was centrifuged at 12,000 rpm at 4° C. for 2 minutes. The precipitate was resuspended in 200 µL PBS, the phage titer was estimated by counting the colonies.

Screening for antigen binders: 96-well plates were coated with 100 µl of 2 µg/mL LAG-3 protein diluted in carbonate buffer solution (pH 9.6) and incubated overnight at 4° C. The plates were washed three times with PBST, 200 µL skimmed milk was added to each well and incubated at 37° C. for 1 hour. Then the plates were washed three times with PBST, and 50 µL phage supernatant and 50 µL 5% skimmed milk were added to each well and incubated at 37° C. for 1 hour. The plates were washed 6 times with PBST, and 100 µL anti-M13 antibody conjugated with HRP (1:5000 in PBS) was added to each well and incubated at 37° C. for 1 hour. The plates were washed 6 times with PBST, and 100 µL TMB per well was added and incubated at 37° C. for 7 minutes. Then 50 µL stop solution was added to each well, and the adsorption at 450 nm was detected in a microplate reader.

### Example 4: Expression and Purification of Single Domain Antibodies

After determination of the sequences of the positive clones, the VHH sequences were cloned into pTT5 vector by PCR. The recombinant single domain antibodies were expressed by ExpiCHO transfection system. Cells were incubated in a shaking incubator at 37° C. for 12 days. Cell culture supernatant was harvested and clarified by centrifugation at 2000 rpm for 10 min, then filtered through a 0.22 um filter. Clarified supernatant was purified using AKTA and MabselectSure (1 ml) column and eluted by 0.2 M Tris-Glycine (pH3.4) buffer. After concentration, the eluted antibodies were further purified through chromatography Superdex 200(GE).

### Example 5: Binding Assays of anti-LAG3 Single Domain Antibodies and Human LAG3 Protein

The binding of the single domain antibodies to recombinant human LAG3 protein (rhLAG3) was examined by Biacore™ assay. The single domain antibodies were captured using an anti-human Fc that was coated on a CM5 chip (Catalog No. BR-1005-30, GE). The coating was carried out according to the manufacturer’s instructions accompanying the kit (Catalog No. BR-1008-39, GE). Then the LAG3-His antigen (Catalog No. 16498-H08H, Sino Biological) was passed through the surface of the CM5 chip. The real-time reaction signals were detected by the Biacore instrument to obtain the binding and dissociation curves thereby to obtain the binding kinetics of the single domain antibodies to rhLAG3 via curve fitting presented in Table 6 below. The chip surface was regenerated after each cycle with 25 mM NaOH followed by HBS-EP wash provided in the kit.

TABLE 6

Binding Kinetics of Single Domain Antibodies to rhLAG3

| | | | |
|---|---|---|---|
| sdAb No | ka (⅟Ms) | kd (⅟s) | KD (M) |
| 1 | 4.77E+06 | 5.05E-08 | N/A |
| 8 | 7.97E+05 | 2.77E-04 | 3.47E-10 |
| 17 | 6.60E+06 | 0.001195 | 1.81E-10 |
| 18 | 2.31E+06 | 3.69E-07 | N/A |
| 20 | 1.71E+06 | 4.29E-04 | 2.52E-10 |
| 21 | 6.41E+06 | 0.00178 | 2.78E-10 |
| 24 | 8.94E+05 | 3.39E-05 | 3.80E-11 |
| 25 | 6.31E+06 | 1.08E-04 | 1.70E-11 |
| 27 | 1.17E+07 | 4.09E-07 | N/A |
| 73 | 5.35E+06 | 9.75E-04 | 1.82E-10 |
| BMS | 6.16E+06 | 5.06E-04 | 8.21E-11 |

The anti-LAG3 antibody produced by Bristol-Myers Squibb (BMS) having the VH and VL sequences shown as follows was used as a positive control in the binding assay. The sequence of the BMS antibody was disclosed in US Pat. Application Publication No. 2011/0150892. Only VH and VL of 25F7 were cloned into PTT5 vector. Another positive control is GS2-2 antibody (W3396-R2-2 disclosed in CN 110305215A). The results demonstrate that the single domain antibodies disclosed herein have strong binding activity and affinity for LAG3 protein.

The amino acid sequence of the VH of the BMS antibody (SEQ ID NO: 77):

QVQLQQWGAGLLKPSETLSLTCAVYGGSFSDYYWNWIRQPPGKGLEWIGE
INHRGSTNSNPSLKSRVTLSLDTSKNQFSLKLRSVTAADTAVYYCAFGYS
DYEYNWFDPWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RWSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK

The amino acid sequence of the VL of the BMS antibody (SEQ ID NO: 78):

EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

The amino acid sequence of the GS2-2 antibody (SEQ ID NO: 79):

QVQLVESGGGWQPGGSLRLSCAASGLTLSQYTMGWFRQAPGKERELVAAI
HWTSSVTDYADSVYGRFTISRDDSKNTGYLQMNSLRAEDTAVYYCAATHY
YTHRGPFDYWGQGTLVTVSS

### Example 6: Epitope Determination of Anti-LAG3 sdAb

The single domain antibodies were captured by anti-Fc coated on a CM5 chip, thereby to capture the anti-LAG3 antibodies (BMS, sdAb 1, 17, 18, 20, 21, 24, 25, 27, 37, and 73) according to the manufacturer’s instructions accompanying the kit (Catalog No. BR-1008-39, GE). Then the LAG3-His antigen (Catalog No. 16498-H08H, Sino Biological) was passed through the surface of the CM5 chip at a flow rate of 25 ug/mL, followed by a blocking antibody (human IgG1 Fc, made in house) pass-through to occupy the remaining unbound sites. Finally, the anti-LAG3 sdAbs (sdAb 1, 17, 18, 20, 21, 24, 25, 27, 37, and 73) as well as the BMS control were passed through the chip surface. The real-time reaction signals were detected by the Biacore instrument to obtain the binding kinetics of the single domain antibodies presented in Table 7 below. The chip surface was regenerated after each cycle with 25 mM NaOH followed by HBS-EP wash provided in the kit.

TABLE 7

Characterization of the single domain antibodies

| | BMS (RU) | 25 | 1 | 17 | 18 | 20 | 21 | 24 | 27 | 37 | 73 | HBS-EP |
|---|---|---|---|---|---|---|---|---|---|---|---|---|
| BMS (RU) | 53 | 33.5 | 27 5 | 14.4 | 21.8 | 24.5 | 27.6 | 28 | 27.7 | 23.2 | 19.6 | 0.4 |
| 25 | 31.7 | 7.2 | 44. 7 | 41 | 39 | 44.6 | 49.3 | 46.4 | 50.9 | 44.1 | 47.6 | 3.9 |
| 1 | 37.4 | 30 | 22 | -6.9 | -10.7 | -14.8 | -114 | -10.6 | 4.9 | -8.4 | -11.6 | 6.1 |
| 17 | 15.2 | 17.9 | -13.6 | -2 | -7.5 | -14.6 | -12.5 | -10.4 | -7 | -13.4 | -17 | 4.9 |
| 18 | 26.9 | 274 | -9.5 | -14.7 | -9.1 | -13.4 | -11.4 | -9.9 | -5.3 | -8.5 | -11.1 | 6.3 |
| 20 | 23.3 | 205 | -9.9 | -7.8 | -12 | -24.2 | -20.1 | -209 | -16.6 | -19.3 | -21.8 | 3.9 |
| 21 | 194 | 179 | -4.7 | -12 | -10.3 | -12.6 | -19.1 | -18.9 | -14.8 | -17 3 | -19.5 | 91 |
| 24 | 30.5 | 33.2 | -8.8 | -13.6 | -11.2 | -12.9 | -12 | -10.1 | -6.6 | -8.5 | -11.3 | 7.1 |
| 27 | 48.2 | 34 | -9.1 | -11.7 | -9.6 | -11.7 | -10.1 | -10.1 | -6.2 | -9.1 | -15.1 | 6 |
| 37 | 26.6 | 31.5 | -10.5 | -14.3 | -11.7 | -13.6 | -118 | -13.4 | -5.8 | -13.3 | -17.6 | 7 |
| 73 | 27 | 9.8 | -7.3 | -12.5 | -10.6 | -117 | -10.2 | -9.6 | -2.2 | -6.9 | -9.7 | 6.3 |

The sdAbs obtained herein bind to epitopes at different locations from the epitope bound by the control BMS antibody. Furthermore, sdAb #25 binds to an epitope at a different location from the epitopes of the remaining sdAbs.

### Example 7: Effect of Single Domain Antibodies on Jurkat T Cell Expressing Human LAG-3

The efficacy of the anti-LAG3 single domain antibodies was determined by the level of IL-2 produced by Jurkat T cells. A Jurkat T cell line stably overexpressing human LAG3 was generated by lentiviral transduction. The flow cytometry results demonstrate that MHCII on Raji B cells can bind to human LAG3, resulting in a significant reduction of the IL-2 production when the Jurkat T cells were activated.

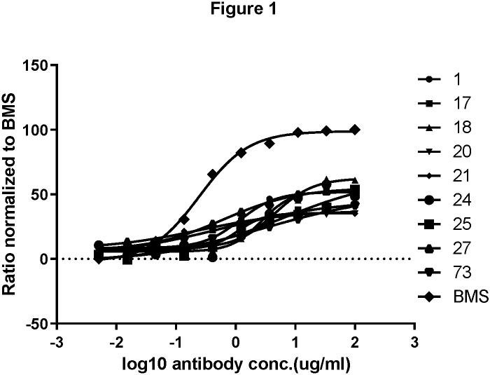

To assess the effect of anti-LAG3 single domain antibodies, Jurkat T cells overexpressing human LAG3 (100,000 per well) and Raji B cells (25,000 per well) were mixed and SEE (50 pg/well) was added (Catalog No. ET404, Toxin Technology). Different anti-LAG3 antibodies (100 µg/mL, 3x serial dilution) were added to the plates. After stimulation at 37° C., 5% CO₂ for 24 hours, IL-2 secretion in the culture supernatant was determined by ELISA. An antagonist antibody restored T-cell function inhibited by overexpressed LAG3 in cell membrane. The EC50 of the single domain antibodies for IL-2 rescue was determined using the hLAG3 Jurkat T system, as shown in FIG. 1, Table 8 and Table 9. The efficacy of BMS antibody is set at 100% as a control.

TABLE 8.

The effects of sdAb Nos. 1, 17, 18, 20, 21, 24, 25, 27, and 73

| | | | | | | | | | | | |
|---|---|---|---|---|---|---|---|---|---|---|---|
| Ab conc.(u g/mL) | LO G Ab | 1 | 17 | 18 | 20 | 21 | 24 | 25 | 27 | 73 | BMS |
| 100 | 2 | 51.2 8989 | 42.2 0053 | 60.9 1913 | 35.0 6577 | 35.0 3029 | 48.2 4586 | 53.7 2583 | 52.8 1271 | 40.9 8897 | 100. 0029 |
| 33.3333 3333 | 1.52 2879 | 54.4 1081 | 38.0 0414 | 61.6 6443 | 34.5 4431 | 38.8 5222 | 57.6 981 | 39.8 1601 | 52.9 1183 | 36.7 6501 | 99.1 4416 |
| 11.1111 1111 | 1.04 5757 | 45.7 3808 | 37.9 5454 | 45.5 0923 | 32.9 9952 | 31.6 5996 | 47.2 0346 | 34.0 6673 | 49.9 6554 | 30.5 8058 | 97.8 4576 |
| 3.70370 3704 | 0.56 8636 | 46.4 4774 | 31.3 6806 | 35.1 2689 | 38.0 2987 | 32.8 1095 | 34.8 6598 | 30.6 3471 | 43.3 6803 | 24.5 1175 | 89.3 9985 |
| 1.23456 7901 | 0.09 1515 | 30.7 4901 | 26.5 4386 | 15.5 2079 | 20.2 845 | 27.7 1135 | 17.7 6784 | 25.0 8632 | 36.5 2821 | 19.5 4434 | 82.1 1682 |
| 0.41152 | - | 14.7 | 22.2 | 6.53 | 14.6 | 24.6 | 1.25 | 9.20 | 27.2 | 15.4 | 65.3 |
| 2634 | -0.38561 | 9935 | 5933 | 0646 | 7389 | 2738 | 7653 | 4503 | 8731 | 8561 | 2968 |
| 0.13717 | - | 14.2 | 18.0 | 5.26 | 6.05 | 21.6 | 10.7 | 2.39 | 17.9 | 6.63 | 30.6 |
| 4211 | -0.86 273 | 1907 | 464 | 7672 | 9972 | 7009 | 8725 | 322 | 9134 | 5718 | 4446 |
| 0.04572 | - | 6.06 | 9.97 | 12.9 | 3.54 | 13.4 | 3.43 | 8.02 | 12.1 | 3.62 | 13.4 |
| 4737 | 1.33 985 | 7809 | 3015 | 031 | 6764 | 3147 | 6769 | 7829 | 4281 | 8846 | 6801 |
| 0.01524 | - | 8.76 | 9.55 | 10.9 | 9.35 | 13.7 | 2.74 | | 13.3 | 3.54 | 2.64 |
| 1579 | -1.81 696 | 921 | 9984 | 2783 | 7327 | 0132 | 3825 | -0.58 952 | 7089 | 6237 | 8129 |
| 0.00508 | - | 8.39 | 5.97 | 7.39 | 8.49 | 10.4 | 10.6 | 3.23 | 5.57 | 0.49 | 0.00 |
| 0526 | -2.29 405 | 6749 | 4866 | 3542 | 0847 | 6315 | 8969 | 5069 | 2847 | 531 | 0609 |

TABLE 9

Single Domain Antibodies Stimulated IL-2 Production

| sdAb | Efficacy | EC50 (nM) |
|---|---|---|
| 1 | 41.40% | 15.67 |
| 17 | 46.50% | 7.72 |
| 18 | 51.90% | 60.65 |
| 20 | 26.90% | 14.24 |
| 21 | 30.98% | 3.41 |
| 24 | 44.70% | 35.81 |
| 25 | 43.40% | 8.12 |
| 27 | 50.22% | 8.49 |
| 73 | 56.39% | 42.6 |
| BMS (25F7) | 100% | 1.33 |

### Example 8: Optimization of in Vitro Efficacy of anti-LAG-3 Single Domain Antibodies

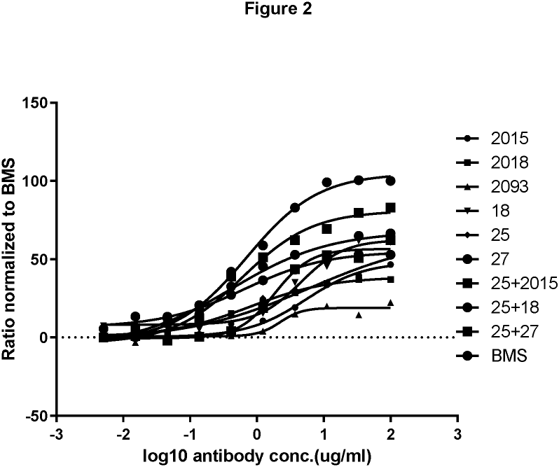

Based on the epitope competition assay, two single domain antibodies binding to two distinct epitopes were combined to improve the in vitro efficacy. The procedure was described in Example 7. As shown in FIG. 2 and Table 10, the combination of two sdAbs binding to different epitopes demonstrated an increase in IL-2 production, resulting enhanced in vitro cell-based efficacy of anti-LAG3 sdAbs.

TABLE 10

Effects of individual sdAb Nos. 2015, 2018, 2093, 18, 25, and 27 in comparison to combination of sdAbs 25 and 2015, combination of sdAbs 25 and 18, and combinationof sdAbs 25 and 27

| | | | | | | | | | | | |
|---|---|---|---|---|---|---|---|---|---|---|---|
| Ab conc.(u g/mL) | LO G Ab | 2015 | 2018 | 2093 | 18 | 25 | 27 | 25+2 015 | 25+1 8 | 25+2 7 | BMS |
| 100 | 2 | 46.6 4049 | 36.9 7342 | 22.1 586 | 60.9 1913 | 53.7 2583 | 52.8 1271 | 62.1 8679 | 66.4 5119 | 82.9 922 | 100. 0002 |
| 33.3333 3333 | 1.52 2879 | 39.7 6946 | 36.9 3102 | 14.4 0014 | 61.6 6443 | 39.8 1601 | 52.9 1183 | 50.7 0897 | 64.9 5188 | 79.7 502 | 100. 4559 |
| 11.1111 1111 | 1.04 5757 | 33.6 1293 | 34.5 7214 | 19.9 9223 | 45.5 0923 | 34.0 6673 | 49.9 6554 | 52.4 7729 | 52.4 6095 | 69.3 2811 | 99.1 8367 |
| 3.70370 3704 | 0.56 8636 | 19.1 4329 | 28.6 0812 | 14.9 1564 | 35.1 2689 | 30.6 3471 | 43.3 6803 | 43.6 0303 | 52.8 7473 | 62.2 5024 | 82.9 4437 |
| 1.23456 7901 | 0.09 1515 | 10.5 8878 | 22.7 8764 | 3.99 5707 | 15.5 2079 | 25.0 8632 | 36.5 2821 | 18.8 9208 | 45.3 6849 | 51.1 9045 | 58.8 0899 |
| 0.41152 2634 | -0.38 561 | 1.79 6063 | 15.3 489 | 1.04 3053 | 6.53 0646 | 9.20 4503 | 27.2 8731 | 3.95 9165 | 32.5 7088 | 39.0 354 | 41.9 8652 |
| 0.13717 4211 | - 0.86 273 | 1.91 6778 | 9.41 4231 | 0.26 6553 | 5.26 7672 | 2.39 322 | 17.9 9134 | 0.43 882 | 14.5 5003 | 10.8 0665 | 20.6 0127 |
| 0.04572 4737 | - 1.33 985 | 0.92 4947 | 2.83 3568 | 5.72 8148 | 12.9 031 | 8.02 7829 | 12.1 4281 | -2.02 771 | 13.2 3569 | -1.96 174 | 8.90 072 |
| 0.01524 1579 | - 1.81 696 | 1.05 5452 | 5.31 6408 | -3.19 18 | 10.9 2783 | -0.58 952 | 13.3 7089 | 0.88 2535 | 1.96 6611 | 4.21 0705 | 0.16 9923 |
| 0.00508 0526 | - 2.29 405 | 0.01 7945 | 0.02 1208 | -0.00 098 | 7.39 3542 | 3.23 5069 | 5.57 2847 | 0.04 7306 | 0 | 0 | 0.02 1777 |

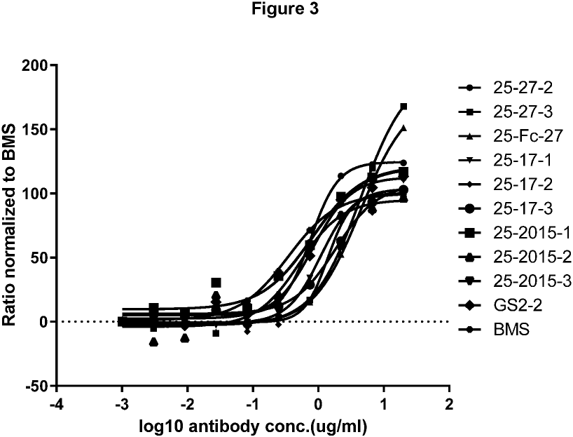

FIG. 3, Table 11 and Table 12 show single domain antibodies, as well as the sdAb fusions, stimulated IL-2 production in the hLAG3 Jurkat T system. The results show that sdAb fusions 25-27-2, 25-17-1, 25-17-2, 25-17-3, 25-2015-1, 25-2015-2, and 25-2015-3 were able to stimulate IL-2 production in a LAG3 T cell/APC bioassay, EC50 was comparable to positive control antibodies BMS and GS2-2 (W3396-R2-2 disclosed in CN 110305215A).

TABLE 11

Effects of various sdAb fusions having different numbers of linkers 25-27-2, 25-27-3, 25-Fc-27, 25-17-1, 25-17-2, 25-17-3, 25-2015-1, 25-2015-2, and 25-2015-3

| | | | | | | | | | | | | |
|---|---|---|---|---|---|---|---|---|---|---|---|---|
| Ab conc.( ug/mL ) | LO G Ab | 25-27-2 | 25-27-3 | 25-Fc-27 | 25-17-1 | 25-17-2 | 25-17-3 | 25-201 5-1 | 25-201 5-2 | 25-201 5-3 | GS2 -2 | BM S |
| 20 | 1.30 103 | 123. 886 6 | 167. 913 5 | 151. 239 6 | 110. 282 9 | 110. 865 6 | 102. 789 5 | 116. 886 4 | 96.5 662 9 | 116. 119 2 | 113. 334 9 | 98.1 641 7 |
| 6.6666 66667 | 0.82 390 9 | 122. 658 2 | 119. 733 | 107. 829 4 | 89.1 905 1 | 85.1 161 3 | 93.2 172 6 | 112. 433 7 | 88.3 454 4 | 111. 695 8 | 104. 544 5 | 96.0 205 |
| 2.2222 22222 | 0.34 678 7 | 113. 537 2 | 58.4 014 8 | 52.5 704 6 | 82.6 537 6 | 77.7 359 3 | 62.7 670 7 | 97.4 552 2 | 83.2 398 3 | 96.8 155 4 | 95.1 092 2 | 93.0 166 5 |
| 0.7407 40741 | - 0.13 033 | 61.9 630 4 | 17.0 077 6 | 15.2 750 7 | 34.0 165 3 | 16.3 504 9 | 28.8 592 8 | 59.7 334 9 | 59.3 953 1 | 59.3 413 1 | 51.3 222 2 | 71.3 030 3 |
| 0.2469 1358 | - 0.60 746 | 17.2 155 1 | 3.52 669 3 | 3.12 876 7 | 6.88 138 8 | - 2.20 551 | 12.0 517 5 | 35.6 057 8 | 20.8 948 | 35.3 719 1 | 18.3 818 | 39.2 723 8 |
| 0.0823 04527 | - 1.08 458 | 6.04 278 6 | - 0.28 916 | - 0.30 931 | - 5.85 478 | - 7.68 195 | 11.6 813 | 10.3 767 1 | - 0.80 208 | 10.3 083 3 | 5.59 347 3 | 16.5 192 3 |
| 0.0274 34842 | - 1.56 17 | 11.1 178 | - 8.89 801 | - 8.06 579 | - 2.73 4 | 11.9 705 5 | 8.90 977 4 | 30.3 743 3 | 20.7 846 1 | 30.1 747 4 | 15.4 021 7 | 11.5 554 7 |
| 0.0091 44947 | - 2.03 882 | 5.39 07 | 2.64 208 8 | 2.33 170 6 | - 0.83 622 | - 0.62 102 | 7.44 168 9 | 7.32 438 3 | - 12.5 08 | 7.27 601 7 | - 3.01 001 | 0.84 987 8 |
| 0.0030 48316 | - 2.51 594 | 3.76 300 9 | - 5.25 377 | - 4.78 237 | 4.10 401 | - 2.51 88 | 5.70 834 | 10.7 857 7 | - 15.5 604 | 10.7 147 3 | - 1.45 934 | - 0.34 475 |
| 0.001016105 | - 2.99 | 0.01 470 | 0.06 734 | 0.01 189 | 0.01 325 | - 0.00 | 0.00 063 | 0.00 029 | 0.00 033 | 0 | 0.02 574 | 0.01 754 |
| | 306 | 3 | | 5 | 2 | 048 | 9 | 2 | 4 | | 4 | 8 |

TABLE 12

Stimulation of T Cell Response by Anti-LAG3 Antibodies and Conjugates

| Antibody/Conjugate | Jurkat T-hLAG3 Assay EC50 (nM) | Efficacy (Normalized to BMS) |
|---|---|---|
| 25-27-2 | 4.82-10.76 | 106.0% |
| 25-17-1 | 10.9 | 106.3% |
| 25-17-2 | 14 | 101.8% |
| 25-17-3 | 18.45 | 102.5% |
| 25-2015-1 | 8.01 | 113.7% |
| 25-2015-2 | 5.13 | 100.9% |
| 25-2015-3 | 8.01 | 113.0% |
| GS2-2 | 6.83-14.3 | 100.1% |
| BMS | 1.67-2.84 | 100% |

### Example 9. Modified sdAb Nos. 17 and 27

Modified sdAb Nos. 17 and 27 were prepared by mutation of one or more amino acids in the CDR regions of sdAb Nos. 17 and 27 that may have a higher possibility of post-translational modification before humanization. The modified sequences are shown in Table 13. The CDR regions are bolded and underlined. The mutations are marked with frames. The CDR regions were calculated by the Kabat numbering method.

TABLE 13

Sequences of Modified sdAb Nos. 17 and 27

| | | | | |
|---|---|---|---|---|
| sdAb name | Sequence | CDR1 | CDR2 | CDR3 |
| | QLQLVESGGGLV QPGGSLRLSCAA SGLTLDGYAIGW FRQAPGKEREGL ACISGSDGSTHYL | GLTLDGYAIG (SEQ ID NO: 98) | CISGSDGSTHYLD SVKG (SEQ ID NO: 102) | RRGSCSGSW TY (SEQ ID NO: 108) |
| | DSVKGRFTISRDN GKNTMALQMNSL QFGDTAIYYCAAR RGSCSGSWTYW GQGTQVTVSS (SEQ ID NO: 3) | | | |
| 17-H1 | QLQLVESGGGLV QPGGSLRLSCAA SGLTLDAYAIGW FRQAPGKEREGL ACISGSDGSTHYL DSVKGRFTISRDN GKNTMALQMNSL QFGDTAIYYCAAR RGSCSGSWTYW GQGTQVTVSS (SEQ ID NO: 80) | GLTLDAYAIG (SEQ ID NO: 99) | CISGSDGSTHYLD SVKG (SEQ ID NO: 102) | RRGSCSGSW TY (SEQ ID NO: 108) |
| 17-H2 | QLQLVESGGGLV QPGGSLRLSCAA SGLTLEGYAIGWF RQAPGKEREGLA CISGSDGSTHYLD SVKGRFTISRDNG KNTMALQMNSLQ FGDTAIYYCAARR GSCSGSWTYWG QGTQVTVSS (SEQ ID NO: 81) | GLTLEGYAIG (SEQ ID NO: 100) | CISGSDGSTHYLD SVKG (SEQ ID NO: 102) | RRGSCSGSW TY (SEQ ID NO: 108) |
| 17-H3 | QLQLVESGGGLV QPGGSLRLSCAA SGLTLDGYAIGW FRQAPGKEREGL ACISGSDASTHYL DSVKGRFTISRDN GKNTMALQMNSL QFGDTAIYYCAAR RGSCSGSWTYW GQGTQVTVSS (SEQ ID NO: 82) | GLTLDGYAIG (SEQ ID NO: 98) | CISGSDASTHYLD SVKG (SEQ ID NO: 103) | RRGSCSGSW TY (SEQ ID NO: 108) |
| 17-H4 | QLQLVESGGGLV QPGGSLRLSCAA SGLTLDGYAIGW FRQAPGKEREGL ACISGSEGSTHYL DSVKGRFTISRDN | GLTLDGYAIG (SEQ ID NO: 98) | CISGSEGSTHYLD SVKG (SEQ ID NO: 104) | RRGSCSGSW TY (SEQ ID NO: 108) |
| | GKNTMALQMNSL QFGDTAIYYCAAR RGSCSGSWTYW GQGTQVTVSS (SEQ ID NO: 83) | | | |
| 17-H5 | QLQLVESGGGLV QPGGSLRLSCAA SGLTLDAYAIGW FRQAPGKEREGL ACISGSDASTHYL DSVKGRFTISRDN GKNTMALQMNSL QFGDTAIYYCAAR RGSCSGSWTYW GQGTQVTVSS (SEQ ID NO:84) | GLTLDAYAIG (SEQ ID NO: 99) | CISGSDASTHYLD SVKG (SEQ ID NO: 103) | RRGSCSGSW TY (SEQ ID NO: 108) |
| 27 | QVQLVESGGGLV QPGGSLRLSCAA SGFTLDYYAIGWF RQAPGKEREGVS CIRSSDGSTYYAD SVKGRFTISSDNT KNTVYLQMNSLK PEDTAVYYCATD RYSDCRGPYENW GQGTQVTVSS (SEQ ID NO: 9) | GFTLDYYAIG (SEQ ID NO: 101) | CIRSSDGSTYYAD SVKG (SEQ ID NO: 105) | DRYSDCRGPY EN (SEQ ID NO: 109) |
| 27-H1 | QVQLVESGGGLV QPGGSLRLSCAA SGFTLDYYAIGWF RQAPGKEREGVS CIRSSDASTYYAD SVKGRFTISSDNT KNTVYLQMNSLK PEDTAVYYCATD RYSDCRGPYENW GQGTQVTVSS (SEQ ID NO: 85) | GFTLDYYAIG (SEQ ID NO: 101) | CIRSSDASTYYAD SVKG (SEQ ID NO: 106) | DRYSDCRGPY EN (SEQ ID NO: 109) |
| 27-H2 | QVQLVESGGGLV QPGGSLRLSCAA SGFTLDYYAIGWF RQAPGKEREGVS CIRSSEGSTYYAD SVKGRFTISSDNT KNTVYLQMNSLK | GFTLDYYAIG (SEQ ID NO: 101) | CIRSSEGSTYYAD SVKG (SEQ ID NO: 107) | DRYSDCRGPY EN (SEQ ID NO: 109) |
| | PEDTAVYYCATD RYSDCRGPYENW GQGTQVTVSS (SEQ ID NO: 86) | | | |

### Example 10. Binding Analysis of Embodiments of Modified sdAb Nos. 17 and 27 to hLAG3-His Protein by ELISA

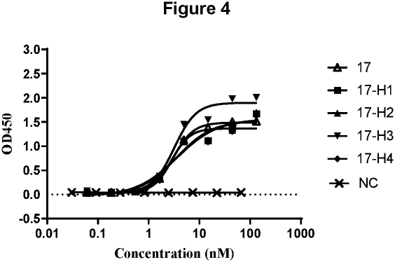

Binding analysis of embodiments of modified sdAb Nos. 17 and 27 to hLAG3-His protein was performed by ELISA as described herein. All the modified 17 sdAbs specifically bound to hLAG3-His protein. SdAb No. 17-H5 showed the highest binding affinity with hLAG3-His protein, and then followed by sdAb No. 17-H3. All the modified 27 sdAbs specifically bound to hLAG3-His protein. sdAb Nos. 27-H2 showed slightly higher binding affinity than sdAb Nos. 27-H1 and sdAb No. 27. See Tables 14-16 and FIGS. 4-6.

Plates were coated with 100 µL per well of Coating Solution, and then covered, and incubated overnight (12-18 hours) at 2-8° C. The wells were aspirated and washed 1 time with >200 µL of Wash buffer per well, inverted and tapped on absorbent paper to remove excess liquid. The plates were blocked with 200 µL per well with Blocking buffer (1×PBS+4% milk) for 1 hour at room temperature, aspirated, inverted, and tapped on absorbent paper to remove excess liquid. Standards and sample dilutions were prepared in Blocking buffer. 100 µL of standards (in duplicate) and samples were pipetted into designated wells. After incubation for 1 hour at room temperature with gentle continual shaking (~500 rpm), the wells were aspirate and washed 3 times with >200 µL of Wash buffer per well, inverted and tapped on absorbent paper to remove excess liquid.

The primary antibody solution was diluted with Blocking buffer. See Table 14 for recommended primary antibody dilution. 100 µL of the primary antibody solution was added into each well and incubated for 2 hours at room temperature with gentle continual shaking (~500 rpm). The wells were aspirated and washed 3 times with >200 µL of Wash buffer per well, inverted and tapped on absorbent paper to remove excess liquid. The working solution of secondary antibody was made with Blocking buffer by diluting the secondary antibody by 2,500 times. 100 µL of secondary antibody working solution was added into each well and incubated for 30 minutes at room temperature. The wells were aspirated and washed 5 times with >200 µL of Wash buffer per well, inverted and tapped on absorbent paper to remove excess liquid. 100 µL of TMB substrate solution (Biopanda, TMB-S-004) was added to each well and incubated for 30 minutes at room temperature. 100 µL of Stop solution (Solarbio, C1058) was added to each well. Absorbance at 450 nm was measured within 30 minutes of adding Stop solution. Results were calculated using a log-log or 4-parameter curve fit.

Materials used are listed below:
1) Coating solution: 1x PBS with LAG3-His (Sino biological, 16498-H08H, 2 ug/ml, 100 ul/well);
2) Blocking buffer: 1×PBS+4% milk (BD,23200);
3) Primary antibodies: sdAb Nos. 17, 27, 17-H1, 17-H2, 17-H3, 17-H4, 27-H1, 27-H2;
4) Secondary antibody: mouse anti-human IgG1 Fc antibody HRP (1:2,500, Invitrogen, MH1715);
5) NC, negative control: anti-PD-L1 antibody KN035 Benemae prepared with sequences shown below:
KN035 HC (SEQ ID NO: 87);

EV QLVESGGGLV QPGGSLRLSC AASGFTFSDS
WIHWVRQAPG KGLEWVAWIS PYGGSTYYAD
SVKGRFTISA DTSKNTAYLQ MNSLRAEDTA VYYCARRHWP
GGFDYWGQGT LVTVSSASTK GPSVFPLAPS SKSTSGGTAA
LGCLVKDYFP EPVTVSWNSG ALTSGVHTFP AVLQSSGLYS
LSSVVTVPSS SLGTQTYICN VNHKPSNTKV DKKVEPKSCD
KTHTCPPCPA PELLGGPSVF LFPPKPKDTL MISRTPEVTC
WVDVSHEDP EVKFNWYVDG VEVHNAKTKP REEQYASTYR
VVSVLTVLHQ DWLNGKEYKC KVSNKALPAP IEKTISKAKG
QPREPQVYTL PPSREEMTKN QVSLTCLVKG FYPSDIAVEW
ESNGQPENNY KTTPPVLDSD GSFFLYSKLT VDKSRWQQGN
VFSCSVMHEA LHNHYTQKSL SLSPGK

KN035 LC (SEQ ID NO: 88):

DIQ MTQSPSSLSA SVGDRVTITC RASQDVSTAV
AWYQQKPGKA PKLLIYSASF LYSGVPSRFS
GSGSGTDFTL TISSLQPEDF ATYYCQQYLY HPATFGQGTK
VEIKRTVAAP SVFIFPPSDE QLKSGTASVV CLLNNFYPRE
AKVQWKVDNA LQSGNSQESV TEQDSKDSTY SLSSTLTLSK
ADYEKHKVYA CEVTHQGLSS PVTKSFNRGE C

TABLE 14

Binding analysis of embodiments of modified sdAb No. 17 to hLAG3-His protein (see FIG. 4 )

| Conc. µg/ml | sdAb No. 17 | sdAb No. 17-H1 | sdAb No. 17-H2 | sdAb No. 17-H3 | sdAb No. 17-H4 | NC |
|---|---|---|---|---|---|---|
| 10 | 1.514 | 1.665 | 1.697 | 2.007 | 1.633 | 0.04 |
| 3.3333333 | 1.502 | 1.368 | 1.331 | 1.981 | 1.307 | 0.036 |
| 1.1111111 | 1.387 | 1.114 | 1.124 | 1.542 | 1.091 | 0.04 |
| 0.3703704 | 1.128 | 1.095 | 1.118 | 1.449 | 1.146 | 0.036 |
| 0.1234568 | 0.334 | 0.375 | 0.339 | 0.363 | 0.292 | 0.041 |
| 0.0411523 | 0.066 | 0.066 | 0.057 | 0.066 | 0.061 | 0.037 |
| 0.0137174 | 0.045 | 0.042 | 0.041 | 0.045 | 0.046 | 0.04 |
| 0.0045725 | 0.039 | 0.041 | 0.041 | 0.073 | 0.048 | 0.043 |
| EC50 (nM) | 3.034 | 3.593 | 3.467 | 3.197 | 2.835 | N/A |

Note: sign ‘~’ means approximately equal to.

TABLE 15

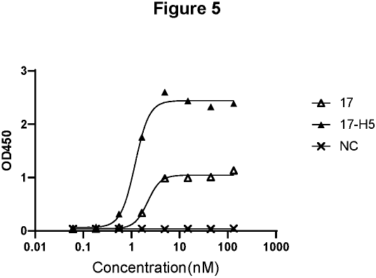

Binding analysis of an embodiment of modified sdAb No. 17 to hLAG3-His protein (see FIG. 5 )

| Conc. nM | NC | sdAb No. 17 | sdAb No. 17-H5 |
|---|---|---|---|
| 133.335 | 0.043 | 1.131 | 2.388 |
| 44.445 | 0.036 | 1.006 | 2.325 |
| 14.815 | 0.041 | 0.99 | 2.435 |
| 4.9383 | 0.034 | 0.983 | 2.601 |
| 1.6461 | 0.04 | 0.333 | 1.756 |
| 0.5487 | 0.035 | 0.06 | 0.315 |
| 0.1829 | 0.04 | 0.042 | 0.07 |
| 0.061 | 0.035 | 0.037 | 0.04 |
| EC50 (nM) | N/A | 2.169 | 1.189 |

Note: sign ‘~’ means approximately equal to.

TABLE 16

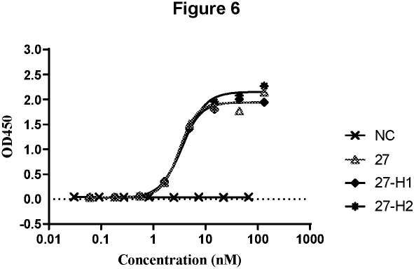

Binding analysis of embodiments of modified sdAb No. 27 to hLAG3-His protein (see FIG. 6 )

| Conc. µg/ml | sdAb No. 27 | sdAb No. 27-H1 | sdAb No. 27-H2 | NC |
|---|---|---|---|---|
| 10 | 2.15 | 1.947 | 2.269 | 0.04 |
| 3.333333 | 1.77 | 2.003 | 2.076 | 0.036 |
| 1.111111 | 1.844 | 1.8 | 1.955 | 0.04 |
| 0.37037 | 1.501 | 1.417 | 1.425 | 0.036 |
| 0.123457 | 0.331 | 0.36 | 0.334 | 0.041 |
| 0.041152 | 0.063 | 0.063 | 0.063 | 0.037 |
| 0.013717 | 0.042 | 0.043 | 0.043 | 0.04 |
| 0.004572 | 0.039 | 0.04 | 0.038 | 0.043 |
| EC50 (nM) | 3.136 | 3.283 | 3.725 | N/A |

### Example 11. Binding Analysis of Embodiments of Modified sdAb Nos. 17 and 27 to 293T-hLAG3 Cells

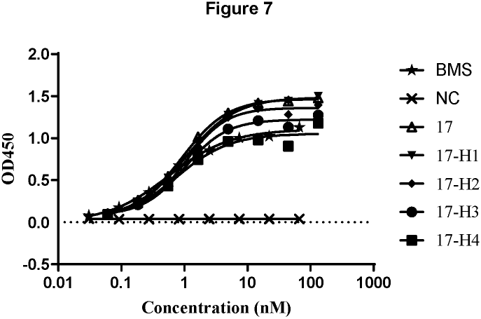

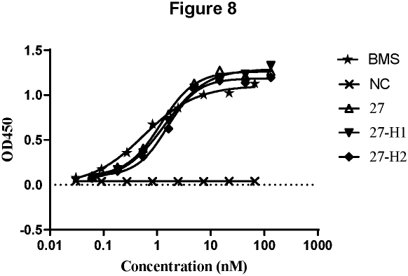

Binding analysis of embodiments of modified sdAb Nos. 17 and 27 to 293T-hLAG3 cells was performed as described herein. All the modified 17 sdAbs specifically bound to 293T-hLAG3 cells. Modified sdAb No. 17-H1 and 17-H2 showed binding affinity to 293T-hLAG3 cells similar to that of sdAb No. 17. All the modified 27 sdAbs specifically bound to 293T-hLAG3 cells. Modified sdAb Nos. 27-H1 and 27-H2 showed binding affinity to 293T-hLAG3 cells similar to that of sdAb No. 27. See Tables 17-18 and FIGS. 7-8.

293T-hLAG3 Cell monolayers were cultured in a 96-well microtiter plate. Each well was treated with stimulants. The cells were then fixed and blocked with Blocking buffer. The Primary antibodies were added. The wells were washed and a HRP-conjugated secondary antibody was added. The wells were washed with 1x PBS and a solution containing TMB (Biopanda, TMB-S-004) was added. The TMB reacted with HRP to change the color thereof from colorless to blue. An acid stop solution (Solarbio, C1058) was then added to stop the reaction and change the blue color to yellow.

Materials are listed below:
1) Cell: 293T-LAG3 (5*10⁵ cell/well) ;
2) Blocking buffer: 1×PBS+3% BSA (Sigma, B2064);
3) Primary antibodies: sdAb Nos. 17, 27, 17-H1, 17-H2, 17-H3, 17-H4, 27-H1, 27-H2.
4) Secondary antibody: Goat anti-human IgG1 Fc antibody, HRP (1:2500, invitrogen, A18817).
5) BMS, positive control: anti-LAG3 antibody Relatimab was prepared in Benemae
6) NC, negative control: anti-PD-L1 antibody KN035 was prepared in Benemae.

TABLE 17

Binding analysis of embodiments of modified sdAb No. 17 to 293T-hLAG3 cells (see FIG. 7 )

| Conc. µg/ml | BMS | sdAb No. 17 | sdAb No. 17-H1 | sdAb No. 17-H2 | sdAb No. 17-H3 | sdAb No. 17-H4 | NC |
|---|---|---|---|---|---|---|---|
| 10 | 1.13 | 1.479 | 1.498 | 1.397 | 1.276 | 1.178 | 0.03 9 |
| 3.333333 | 1.02 4 | 1.44 | 1.44 | 1.282 | 1.132 | 0.905 | 0.03 9 |
| 1.111111 | 1.00 4 | 1.417 | 1.403 | 1.354 | 1.21 | 0.979 | 0.03 8 |
| 0.370370 4 | 0.85 8 | 1.298 | 1.275 | 1.248 | 1.11 | 0.962 | 0.03 8 |
| 0.123456 8 | 0.67 5 | 1.015 | 0.945 | 0.9 | 0.818 | 0.745 | 0.03 7 |
| 0.041152 3 | 0.35 7 | 0.543 | 0.527 | 0.458 | 0.43 | 0.432 | 0.03 6 |
| 0.013717 4 | 0.17 6 | 0.259 | 0.242 | 0.238 | 0.208 | 0.232 | 0.03 7 |
| 0.004572 5 | 0.08 1 | 0.104 | 0.101 | 0.105 | 0.096 | 0.092 | 0.04 3 |
| EC50 (nM) | 0.56 67 | 0.9039 | 1.011 | 1.07 | 1.013 | 0.7585 | N/A |

TABLE 18

Binding analysis of embodiments of modified sdAb No. 27 to 293T-hLAG3 cells (see FIG. 8 )

| Conc. µg/ml | BMS | sdAb No. 27 | sdAb No. 27-H1 | sdAb No. 27-H2 | NC |
|---|---|---|---|---|---|
| 10 | 1.13 | 1.264 | 1.33 | 1.198 | 0.039 |
| 3.333333 | 1.024 | 1.21 | 1.235 | 1.14 | 0.039 |
| 1.111111 | 1.004 | 1.277 | 1.169 | 1.161 | 0.038 |
| 0.3703704 | 0.858 | 1.131 | 1.08 | 1.067 | 0.038 |
| 0.1234568 | 0.675 | 0.79 | 0.719 | 0.626 | 0.037 |
| 0.0411523 | 0.357 | 0.408 | 0.359 | 0.314 | 0.036 |
| 0.0137174 | 0.176 | 0.198 | 0.171 | 0.152 | 0.037 |
| 0.0045725 | 0.081 | 0.097 | 0.076 | 0.075 | 0.043 |
| EC50 (nM) | 0.5667 | 1.18 | 1.396 | 1.55 | N/A |

### Example 12. Humanization of sdAb Nos. 17, 25, 27, and Modified sdAb Nos. 17 and 27

Antibody humanization could decrease the immunogenicity of monoclonal antibodies and improve their activation of the human immune system by replacing non-human antibody frameworks with human ones. It is a very important step in the therapeutic antibody discovery process.

Humanization process was carried out as follows: 1). Hotspot analysis and removal; 2).Determination of the canonical structures of the parental VHNL based on Kabat numbering method; 3). Selection of the best germline framework based on homology; 4). Selection of the J region based on homology; 5). Identification of residues critical in loop conformation and interface; 6). Identification of residues within 5 Å of CDR binding region by modeling; 7). Vector construction of multiple humanized Ab with various back-mutation; 8). Expression and purification of humanized sdAbs; 9). Characterization of humanized Abs by SEC, SDS-PAGE; and 10). Primary screening by ELISA. The CDR regions were calculated by the Kabat numbering method.

TABLE 19.

Sequences of Humanized sdAb Nos. 25, and Humanized Modified sdAb Nos. 17 and 27

| | | | | |
|---|---|---|---|---|
| sdAb name | Sequence | CDR1 | CDR2 | CDR3 |
| 17 | QLQLVESGGGLVQPGGSLRLS CAASGLTLDGYAIGWFRQAPG KEREGLACISGSDGSTHYLDSV KGRFTISRDNGKNTMALQMNSL QFGDTAIYYCAARRGSCSGSW TYWGQGTQVTVSS (SEQ ID NO:3) | GLTLDGYAI G (SEQ ID NO: 98) | CISGSDGS THYLDSVK G (SEQ ID NO: 102) | RRGSCSGS WTY (SEQ ID NO: 108) |
| 17-H5 | QLQLVESGGGLVQPGGSLRLS CAASGLTLDAYAIGWFRQAPG KEREGLACISGSDASTHYLDSV KGRFTISRDNGKNTMALQMNSL QFGDTAIYYCAARRGSCSGSW TYWGQGTQVTVSS (SEQ ID NO: 84) | GLTLDAYAI G (SEQ ID NO: 99) | CISGSDAS THYLDSVK G (SEQ ID NO: 103) | RRGSCSGS WTY (SEQ ID NO: 108) |
| 17-H5B1 | EVQLVESGGGLVQPGGSLRLS CAASGLTLDAYAIGWFRQAPG KEREGVACISGSDASTHYLDSV KGRFTISRDNSKNTLYLQMNSL RAEDTAVYYCAARRGSCSGSW TYWGQGTTVTVSS (SEQ ID NO: 89) | GLTLDAYAI G (SEQ ID NO: 99) | CISGSDAS THYLDSVK G (SEQ ID NO: 103) | RRGSCSGS WTY (SEQ ID NO: 108) |
| 17-H5B2 | QVQLVESGGGLVQPGGSLRLS CAASGLTLDAYAIGWFRQAPG KEREGVACISGSDASTHYLDSV KGRFTISRDNSKNTMYLQMNSL RAEDTAVYYCAARRGSCSGSW TYWGQGTTVTVSS (SEQ ID | GLTLDAYAI G (SEQ ID NO: 99) | CISGSDAS THYLDSVK G (SEQ ID NO: 103) | RRGSCSGS WTY (SEQ ID NO: 108) |
| | NO: 90) | | | |
| 17-H5B3 | QLQLVESGGGLVQPGGSLRLS CAASGLTLDAYAIGWFRQAPG KEREGLACISGSDASTHYLDSV KGRFTISRDNSKNTMYLQMNSL RAEDTAVYYCAARRGSCSGSW TYWGQGTTVTVSS (SEQ ID NO: 91) | GLTLDAYAI G (SEQ ID NO: 99) | CISGSDAS THYLDSVK G (SEQ ID NO: 103) | RRGSCSGS WTY (SEQ ID NO: 108) |
| 25 | QLQLVESGGGLVQAGGSLRLS CAASGSIESFSFMGWYRQAPG KQRELVAGFTGAASLSFSTYTD AVKGRFTISRDNAKNTVYLQMN SLKPEDTAVYFCNVDPWSQSW PGRYWGQGTQVTVSS (SEQ ID NO: 8) | GSIESFSF MG (SEQ ID NO: 110) | GFTGAASL SFSTYTDA VKG (SEQ ID NO: 111) | DPWSQSW PGRY (SEQ ID NO: 112) |
| 25-B5 | QVQLVESGGGLVKPGGSLRLS CAASGSIESFSFMGWYRQAPG KQREWVAGFTGAASLSFSTYT DAVKGRFTISRDNAKNSLYLQM NSLRAEDTAVYYCNVDPWSQS WPGRYWGQGTTVTVSS (SEQ ID NO: 92) | GSIESFSF MG (SEQ ID NO: 110) | GFTGAASL SFSTYTDA VKG (SEQ ID NO: 111) | DPWSQSW PGRY (SEQ ID NO: 112) |
| 25-B6 | QLQLVESGGGLVKPGGSLRLSC AASGSIESFSFMGWYRQAPGK QREWVAGFTGAASLSFSTYTD AVKGRFTISRDNAKNSVYLQMN SLRAEDTAVYYCNVDPWSQSW PGRYWGQGTTVTVSS (SEQ ID NO: 93) | GSIESFSF MG (SEQ ID NO: 110) | GFTGAASL SFSTYTDA VKG (SEQ ID NO: 111) | DPWSQSW PGRY (SEQ ID NO: 112) |
| 25-B7 | QLQLVESGGGLVKPGGSLRLSC AASGSIESFSFMGWYRQAPGK QRELVAGFTGAASLSFSTYTDA VKGRFTISRDNAKNSVYLQMNS LRAEDTAVYFCNVDPWSQSWP GRYWGQGTTVTVSS (SEQ ID NO: 94) | GSIESFSF MG (SEQ ID NO: 110) | GFTGAASL SFSTYTDA VKG (SEQ ID NO: 111) | DPWSQSW PGRY (SEQ ID NO: 112) |
| 27 | QVQLVESGGGLVQPGGSLRLS CAASGFTLDYYAIGWFRQAPGK EREGVSCIRSSDGSTYYADSVK GRFTISSDNTKNTVYLQMNSLK | GFTLDYYAI G (SEQ ID NO: 101) | CIRSSDGS TYYADSVK G (SEQ ID | DRYSDCRG PYEN (SEQ ID NO: 109) |
| | PEDTAVYYCATDRYSDCRGPYE NWGQGTQVTVSS (SEQ ID NO: 9) | | NO: 105) | |
| 27-H1 | QVQLVESGGGLVQPGGSLRLS CAASGFTLDYYAIGWFRQAPGK EREGVSCIRSSDIAISTYYADSVK GRFTISSDNTKNTVYLQMNSLK PEDTAVYYCATDRYSDCRGPYE NWGQGTQVTVSS (SEQ ID NO: 85) | GFTLDYYAI G (SEQ ID NO: 101) | CIRSSDAST YYADSVKG (SEQ ID NO: 106) | DRYSDCRG PYEN (SEQ ID NO: 109) |
| 27-H1B1 | EVQLVESGGGLVQPGGSLRLS CAASGFTLDYYAIGWFRQAPGK EREWVSCIRSSDASTYYADSVK GRFTISSDNSKNTLYLQMNSLR AEDTAVYYCATDRYSDCRGPYE NWGQGTMVTVSS (SEQ ID NO: 95) | GFTLDYYAI G (SEQ ID NO: 101) | CIRSSDAST YYADSVKG (SEQ ID NO: 106) | DRYSDCRG PYEN (SEQ ID NO: 109) |
| 27-H1B2 | QVQLVESGGGLVQPGGSLRLS CAASGFTLDYYAIGWFRQAPGK EREWVSCIRSSDASTYYADSVK GRFTISSDNSKNTLYLQMNSLR AEDTAVYYCATDRYSDCRGPYE NWGQGTMVTVSS (SEQ ID NO: 96) | GFTLDYYAI G (SEQ ID NO: 101) | CIRSSDAST YYADSVKG (SEQ ID NO: 106) | DRYSDCRG PYEN (SEQ ID NO: 109) |
| 27-H1B3 | QVQLVESGGGLVQPGGSLRLS CAASGFTLDYYAIGWFRQAPGK EREGVSCIRSSDASTYYADSVK GRFTISSDNSKNTVYLQMNSLR AEDTAVYYCATDRYSDCRGPYE NWGQGTMVTVSS (SEQ ID NO: 97) | GFTLDYYAI G (SEQ ID NO: 101) | CIRSSDAST YYADSVKG (SEQ ID NO: 106) | DRYSDCRG PYEN (SEQ ID NO: 109) |

Note: The CDR regions were calculated by Kabat numbering method and underlined in bold. Mutated hotspot amino acids (AAs) were marked with frames.

### Example 13. Binding Analysis of Embodiments of Humanized sdAb Nos. 17-H5, 25, and 27-H1 to hLAG3-His Protein by ELISA

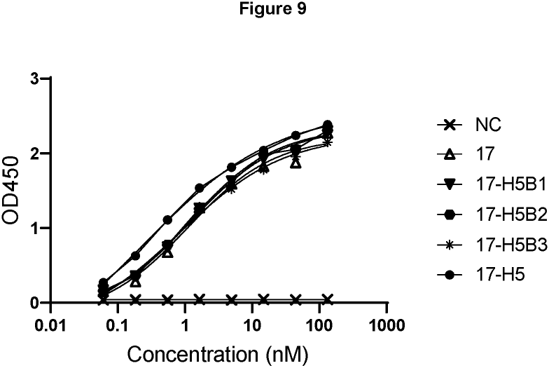

Binding analysis of embodiments of humanized sdAb Nos. 17-H5, 25, and 27-H1 to hLAG3-His protein was performed by ELISA as described in Example 10. All the humanized sdAb Nos. 17-H5 specifically bound to hLAG3-His protein, and showed binding affinity to hLAG3-His protein similar to that of SdAb No. 17. All humanized sdAb No. 17-H5 had slightly lower binding affinity to hLAG3-His protein than SdAb No. 17-H5. Among the several examples of humanized sdAb No. 25, sdAb No. 25-B7 specifically bound to hLAG3-His protein and showed higher binding affinity to hLAG3-His protein than sdAb No. 25, while sdAb Nos. 25-B5 and 25-B6 showed low binding affinity to hLAG3-His protein. Among the several examples of humanized sdAb No. 27-H1, sdAb No. 27-H1B3 specifically bound to hLAG3-His protein and showed higher binding affinity to hLAG3-His protein than sdAb No. 27, while sdAb Nos. 27-H1B1 and 27-H1B2 showed low binding affinity to hLAG3-His protein. See Tables 20-21 an FIGS. 9-11.

The ELISA assay performed herein was similar to the ELISA assay described in Example 10 except for the primary antibodies tested were sdAb Nos. 17, 17-H5, 17-H5B1, 17-H5B2, 17-H5B3, 25, 25-B5, 25-B6, 25-B7, 27, 27-H1B1, 27-H1B2, and 27-H1B3; and anti-PD-L1 antibody KN035 was used as NC.

TABLE 20

Binding analysis of embodiments of humanized sdAb No. 17-H5 to hLAG3-His protein (see FIG. 9 )

| Conc.n M | sdAb No. 17 | sdAb No. 17-H5B1 | sdAb No. 17-H5B2 | sdAb No. 17-H5B3 | sdAb No. 17-H5 | NC |
|---|---|---|---|---|---|---|
| 133.33 5 | 2.27 | 2.298 | 2.306 | 2.149 | 2.385 | 0.04 3 |
| 44.445 | 1.875 | 2.036 | 2.053 | 1.957 | 2.239 | 0.04 1 |
| 14.815 | 1.827 | 1.934 | 1.983 | 1.782 | 2.038 | 0.04 6 |
| 4.9383 | 1.59 | 1.628 | 1.138* | 1.52 | 1.813 | 0.03 9 |
| 1.6461 | 1.27 | 1.265 | 1.258 | 1.269 | 1.536 | 0.04 5 |
| 0.5487 | 0.677 | 0.746 | 0.773 | 0.759 | 1.108 | 0.03 8 |
| 0.1829 | 0.284 | 0.358 | 0.365 | 0.364 | 0.628 | 0.04 2 |
| 0.061 | 0.153 | 0.17 | 0.161 | 0.149 | 0.27 | 0.04 2 |
| EC50 (nM) | 1.119 | 1.206 | 1.177 | 0.8340 | 0.2453 | N/A |

Note: sign ‘~’ means approximately equal to.

TABLE 21

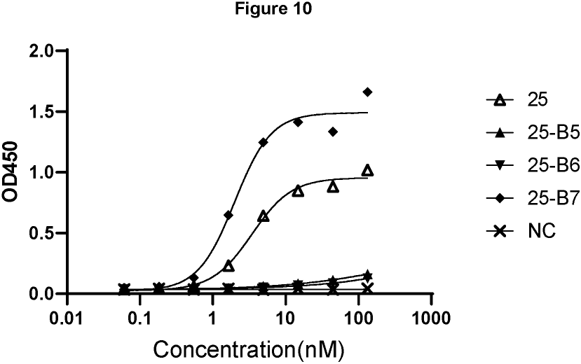

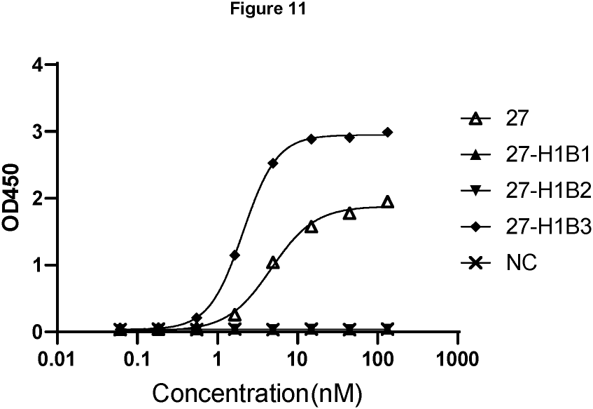

Binding analysis of embodiments of humanized sdAb No. 25, and humanized sdAb No. 27-H1 to hLAG3-His protein (see FIGS. 10-11 )

| Conc . nM | sdAb No. 25 | sdAb No. 25-B5 | sdAb No. 25-B6 | sdAb No. 25-B7 | sdAb No. 27 | sdAb No. 27-H1B1 | sdAb No. 27-H1B2 | sdAb No. 27-H1B3 | N C |
|---|---|---|---|---|---|---|---|---|---|
| 133.3 35 | 1.019 | 0.163 | 0.128 | 1.661 | 1.947 | 0.042 | 0.041 | 2.989 | 0. 04 3 |
| 44.44 5 | 0.882 | 0.114 | 0.085 | 1.334 | 1.775 | 0.036 | 0.037 | 2.907 | 0. 03 6 |
| 14.81 5 | 0.85 | 0.084 | 0.069 | 1.413 | 1.575 | 0.04 | 0.04 | 2.884 | 0. 04 1 |
| 4.938 3 | 0.643 | 0.062 | 0.053 | 1.245 | 1.042 | 0.037 | 0.037 | 2.526 | 0. 03 4 |
| 1.646 1 | 0.233 | 0.048 | 0.044 | 0.648 | 0.257 | 0.04 | 0.04 | 1.148 | 0. 04 |
| 0.548 7 | 0.06 | 0.038 | 0.037 | 0.132 | 0.059 | 0.036 | 0.036 | 0.212 | 0. 03 5 |
| 0.182 9 | 0.042 | 0.04 | 0.039 | 0.057 | 0.042 | 0.04 | 0.04 | 0.057 | 0. 04 |
| 0.061 | 0.037 | 0.036 | 0.037 | 0.038 | 0.037 | 0.037 | 0.036 | 0.038 | 0. 03 5 |
| EC50 (nM) | 3.450 | 452.1 | ∼ 1550184 | 1.996 | 4.695 | ~ 149.4 | ~ 488.7 | 2.081 | N/ A |
| | | | 80 | | | | | | |

Note: sign ‘~’ means approximately equal to.

### Example 14. Binding Analysis of Embodiments of Humanized sdAb No. 25 to 293T-LAG3 Cells

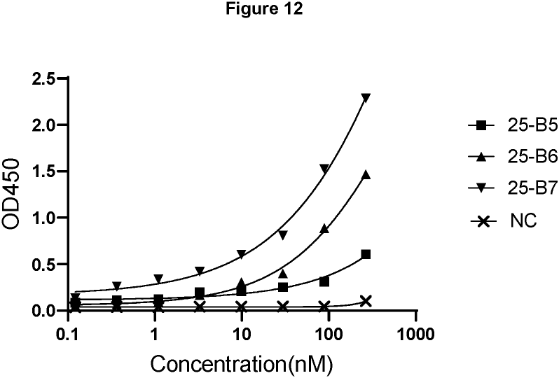

Binding analysis of embodiments of humanized sdAb No. 25 to 293T-hLAG3 cells was performed as described in Example 11. SdAb No. 25-B7 showed higher binding affinity to 293T-LAG3 cells than sdAb No. 25-B6 and 25-B5. See Table 22 and FIG. 12.

TABLE 22

Binding analysis of embodiments of humanized sdAbs No. 25 to 293T-LAG3 cells (see FIG. 12 )

| Conc. nM | sdAb No. BMS | sdAb No. 25-B5 | sdAb No. 25-B6 | sdAb No. 25-B7 | NC |
|---|---|---|---|---|---|
| 266.67 | 0.393 | 0.608 | 1.468 | 2.287 | 0.103 |
| 88.89 | 0.296 | 0.311 | 0.888 | 1.526 | 0.044 |
| 29.63 | 0.262 | 0.254 | 0.399 | 0.809 | 0.044 |
| 9.877 | 0.214 | 0.21 | 0.306 | 0.604 | 0.042 |
| 3.292 | 0.223 | 0.196 | 0.166 | 0.419 | 0.042 |
| 1.097 | 0.138 | 0.124 | 0.095 | 0.337 | 0.039 |
| 0.366 | 0.158 | 0.113 | 0.07 | 0.258 | 0.039 |
| 0.122 | 0.106 | 0.086 | 0.056 | 0.134 | N/A |

### Example 15. Binding Analysis of Humanized sdAb Nos. 25-B7 and 27-H1B3 to 293T-LAG3 Cells by FACS

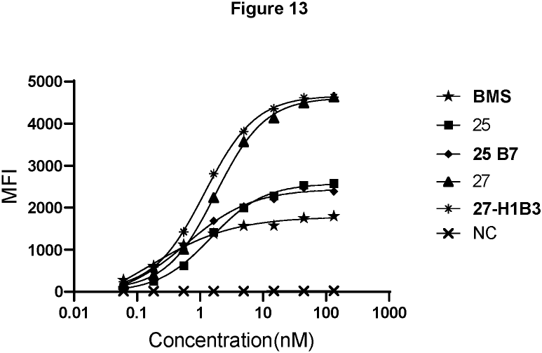

Binding analysis of humanized sdAb Nos. 25-B7 and 27-H1B3 to 293T-hLAG3 cells was performed as described herein. All the tested sdAbs specifically bound to 293T-hLAG3 cells. Humanized sdAb No. 25-B7 showed higher binding affinity to 293T-hLAG3 cell than sdAb No. 25; and humanized sdAb No. 27-H1 B3 showed slightly higher binding affinity to 293T-hLAG3 than sdAb No. 27. See Table 23 and FIG. 13.

FACS procedure performed was set forth below:

1) 293T-hLAG3 cells were digested and count with trypan blue staining in the Counter Star, the viable cell density was 4.16 × 10⁶ cells/ml, the volume was 15 mL, and the cell viability was 93.03%.

2) The 293T-LAG3 cells were washed three times with PBS, and resuspended to 5 × 10⁶ cells/mL with a 1xPBS solution containing 3% BSA.

3) According to the platemap, 100 µl cell suspension was added to each well of a 96-well plate.

4) Antibody samples with desired antibody concentrations were prepared with 3% BSA/1xPBS.

5) The prepared antibody samples were added to the 96-well plate according to the platemap, the cells were resuspended and incubate at 4° C. for 1 hour.

6) The cells were centrifuged at 400 g for 5 minutes and washed 4 times.

7) 3% BSA/1xPBS was used to dilute Goat polyclonal Secondary Antibody to Human IgG-Fc (DyLight® 650) to the desired concentration (1:200), in which the cells were resuspended and incubated at 4° C. for 1 hour.

8) The cells were centrifuged at 400 g for 5 minutes, and washed 3 times.

9) The cells were resuspended in 200 µl of 3% BSA/1xPBS, and performed flow cytometric testing by Shsti Biotech Inc.

The conditions of the FACS procedure performed were:
1) Cells: 293T-LAG3 (5×10⁵/well);
2) Blocking Buffer: 1×PBS+ 3% BSA;
3) Primary antibodies: sdAb Nos. 25, 25-B7, 27 and 27-H1B3;
4) The BMS antibody descried herein as the positive control;
5) Secondary antibody: Goat polyclonal Secondary Antibody to Human IgG-Fc (DyLight® 650) (1: 200).

TABLE 23

Binding analysis of embodiments of sdAbs Nos. 25-B7 and 27-H1B3 to 293T-LAG3 cells by FACS (see FIG. 13 )

| Conc. nM | BMS | sdAb No. 25 | sdAb No. 25-B7 | sdAb No. 27 | sdAb No. 27-H1B3 | NC |
|---|---|---|---|---|---|---|
| 133.335 | 1798 | 2575 | 2384 | 4632 | 4632 | 23.2 |
| 44.445 | 1752 | 2537 | 2477 | 4491 | 4618 | 17.4 |
| 14.815 | 1574 | 2280 | 2207 | 4133 | 4355 | 18 |
| 4.9383 | 1570 | 1997 | 2026 | 3568 | 3816 | 16.1 |
| 1.6461 | 1381 | 1412 | 1687 | 2240 | 2809 | 16.1 |
| 0.5487 | 1121 | 618 | 1112 | 1006 | 1424 | 15.4 |
| 0.1829 | 616 | 247 | 487 | 393 | 585 | 14.8 |
| 0.061 | 280 | 99.2 | 191 | 163 | 234 | 13.5 |
| EC50 (nM) | 0.1533 | 1.482 | 0.6002 | 1.770 | 1.181 | N/A |

What is claimed is:

1. A single domain anti-LAG3 antibody that specifically binds to human LAG3, wherein the single domain antibody comprises an amino acid sequence selected from the group consisting of SEQ ID NOs: 1-19, 80-86, and 89-97, or an amino acid sequence that is at least 85%, at least 90%, at least 95%, at least 98%, or at least 99% identical to a sequence selected from the group consisting of SEQ ID NOs: 1-19, 80-86, and 89-97.

2. A single domain anti-LAG3 antibody that specifically binds to human LAG3, wherein the single domain antibody comprises a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, and/or a CDR3 having an amino acid sequence selected from the group consisting of SEQ ID NOs: 42-56, 108, 109, and 112.

3. The single domain anti-LAG3 antibody of claim 2, wherein:
the single domain antibody comprises a CDR1 having an amino acid sequence selected from the group consisting of SEQ ID NOs: 20-31, 98-101, and 110, a CDR2 having an amino acid sequence selected from the group consisting of SEQ ID NOs: 32-41, 102-107, and 111, and/or a CDR3 having an amino acid sequence selected from the group consisting of SEQ ID NOs: 42-56, 108, 109, and 112; and
one or more DG of one or more CDRs selected from CDR1, CDR2, or CDR3 are mutated to DA and/or EG, and/or one or more NG of one or more CDRs selected from CDR1, CDR2, or CDR3 are mutated to NA and/or QG, wherein D is aspartic acid, G is glycine, A is alanine, E is glutamic acid, N is asparagine, and Q is glutamine.

4. A single domain antibody fusion comprising two or more single domain anti-LAG3 antibodies of claim 1.

5. The single domain antibody fusion of claim 4, further comprising one or more G4S linkers.

6. The single domain antibody fusion of claim 4, further comprising an Fc region or a fragment thereof.

7. The single domain antibody fusion of claim 4, wherein the two or more single domain antibodies bind to epitopes located at different locations or different functional domains of the LAG3 antigen.

8. A single domain antibody fusion comprising the amino acid sequence selected from the group consisting of SEQ ID NOs: 58-76, or an amino acid sequence that is at least 85%, at least 90%, at least 95%, at least 98%, or at least 99% identical to a sequence selected from the group consisting of SEQ ID NOs: 58-76.

9. A pharmaceutical composition comprising a therapeutically effective amount of one or more single domain anti-LAG3 antibodies of claim 1.

10. The pharmaceutical composition of claim 9, wherein the two or more single domain anti-LAG3 antibodies bind to epitopes located at different locations or functional domains of the LAG3 antigen.

11. Use of the single domain antibody of claim 1 in manufacturing a medication for treating or preventing lymphoma, non-small cell lung cancer (NSCLC), gastric cancer, hepatocellular carcinoma, renal cell carcinoma, bladder cancer, squamous cell carcinoma of the head and neck, melanoma, or glioblastoma in a subject.

12. A method of treating or preventing one or more conditions of a subject comprising administering to the subject a therapeutically effective amount of one or more single domain antibodies of claim 1, wherein the one or more conditions are selected from the group consisting of lymphoma, non-small cell lung cancer (NSCLC), gastric cancer, hepatocellular carcinoma, renal cell carcinoma, bladder cancer, squamous cell carcinoma of the head and neck, melanoma, and glioblastoma.

13. The method of claim 12, comprising administering to the subject two or more single domain antibodies of claim 1.

14. The method of claim 13, wherein the two or more single domain antibodies are administered simultaneously or sequentially.

15. A method of treating or preventing one or more conditions of a subject comprising administering to the subject a therapeutically effective amount of the pharmaceutical composition of claim 9, wherein the one or more conditions are selected from the group consisting of lymphoma, non-small cell lung cancer (NSCLC), gastric cancer, hepatocellular carcinoma, renal cell carcinoma, bladder cancer, squamous cell carcinoma of the head and neck, melanoma, and glioblastoma.


SEQUENCE LISTING (112 sequences)

SEQ ID NO: 1 - protein, 121 aa, synthetic construct
  REGION 1..121: note = sdAb
QLQLVESGGGLVQAGGSLRLSCAASGFTFDDYAIGWFRQAPGKEREGVSCIRSSDGSTYYADSVKGRFTISSDNAKNTVYLQMNSLKPEDTAVYYCAKDNYSDCRGPYHYWGQGTQVTVSS

SEQ ID NO: 2 - protein, 125 aa, synthetic construct
  REGION 1..125: note = sdAb
QLQLVESGGGLVQPGGSLRLSCAASGFTLDYYAIGWFRQAPGKEREGVSCISSSDGSTYYADSVKGRFTISRDNAKNTVYLQMNSLKPEDTAIYYCARDSLSGSGYGCRGPYDYWGQGTQVTVSS

SEQ ID NO: 3 - protein, 120 aa, synthetic construct
  REGION 1..120: note = sdAb
QLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSS

SEQ ID NO: 4 - protein, 124 aa, synthetic construct
  REGION 1..124: note = sdAb
QVQLVESGGGLVQPGGSLRLSCAASGFTLNEHAIGWFRRAPGKEREGVSCISSSDGSTYYHDSVKGRFNMSRDDAKNSVYLQMSSLKPEDTAIYYCAAALGRDCQGGQWKTSSWGQGTQVTVSS

SEQ ID NO: 5 - protein, 120 aa, synthetic construct
  REGION 1..120: note = sdAb
QVQLVESGGGLVQPGGSLRLSCAASGLTLDGYAVGWFRQAPGKDREGLSCISGSDGSTHYLDSVKGRFTISRDNAKSTMALQMNSLQFGDTAIYYCAARRGSCSGSWTYWGQGTQVTVSS

SEQ ID NO: 6 - protein, 120 aa, synthetic construct
  REGION 1..120: note = sdAb
QLQLVESGGGLVQPGGSLRLSCAASGLTLDGYAIAWFRQAPGKEREGLSCISGTDGSTHYLDSVKGRFTISRDNGKNTMALQMNSLQFGDTAIYYCAARRGSCSGSWTYWGQGTQITVSS

SEQ ID NO: 7 - protein, 124 aa, synthetic construct
  REGION 1..124: note = sdAb
QLQLVESGGGLVQPGGSLRLSCAASGFTLNEHAIGWFRQAPGKEREGVSCISSSDGSTYYHDSVKGRFTMSRDDAKNTVYLQMNSLKAEDTAIYYCAAALGRDCQGGQWKTSSWGQGTQVTVSS

SEQ ID NO: 8 - protein, 122 aa, synthetic construct
  REGION 1..122: note = sdAb
QLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSS

SEQ ID NO: 9 - protein, 121 aa, synthetic construct
  REGION 1..121: note = sdAb
QVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSS

SEQ ID NO: 10 - protein, 121 aa, synthetic construct
  REGION 1..121: note = sdAb
QVQLVESGGGLVQPGGSLKLSCTASGFTLDYYAIAWFRQAPGKEREAVSCISSSDGSTRYADSVKGRFTISRDNAKNTVYLQMNSLKPEDTAVYYCAADPYGDCRGPYDYWGQGTQVTVSS

SEQ ID NO: 11 - protein, 121 aa, synthetic construct
  REGION 1..121: note = sdAb
QLQLVESGGGLVQAGGSLRLSCAASGFFFDDYAIAWFRQAPGKDREGVSCISSSDGSTVYADSVKGRFTSSRDNAKDTVDLQMNSLKPEDTAVYYCAVRRGYGCSGPWHYWGQGTQVTVSS

SEQ ID NO: 12 - protein, 124 aa, synthetic construct
  REGION 1..124: note = sdAb
QVQLVESGGGLVQPGGSLRLSCAASGFTLNEHAIGWFRQAPGKEREGVSCISSSDGSTYYHDSVKGRFTMSRDDAKNTVYLQMNSLKPEDTAIYYCAAALGRDCQGGQWKTDSWGQGTQVTVSS

SEQ ID NO: 13 - protein, 124 aa, synthetic construct
  REGION 1..124: note = sdAb
QLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSS

SEQ ID NO: 14 - protein, 121 aa, synthetic construct; identical to SEQ ID NO: 10
  REGION 1..121: note = sdAb

SEQ ID NO: 15 - protein, 121 aa, synthetic construct
  REGION 1..121: note = sdAb
QLQLVESGGGLVQPGGSLRLSCAASGFTLDSYAIGWFRQAPGKEREAVSCITSSDGRTYYADSVKGRFTISRDNAKNTVYLQMNSLKPEDTAVYYCAADRWQTCTGPYDHWGQGTQVTVSS

SEQ ID NO: 16 - protein, 127 aa, synthetic construct
  REGION 1..127: note = sdAb
QVQLVESGGGLVQPGGSLRLSCAASGFTLDNYAIGWFRQAPGKEREGVSCISRNAGSTKYADSVKGRFTISRDNAKNTVYLQMNSLKPEDTAVYYCAARVPDYGDITDCGGKWDDYWGPGTQVTVSS

SEQ ID NO: 17 - protein, 119 aa, synthetic construct
  REGION 1..119: note = sdAb
QLQLVESGGGLVQTGGSLRLSCAASGFIFDDYSIGWFRQAPGRHREVVSCISSSDTKYADSAKGRFTISRNNAEDTVYLQMNSLKPEDTAVYYCAARSGDGCNGRWDYWGQGTQVTVSS

SEQ ID NO: 18 - protein, 123 aa, synthetic construct
  REGION 1..123: note = sdAb
QVQLVESGGGLVQPGGSLTLSCVASGFTLDYYTVGWFRQVPGKEREGISCISSSDGSTRYADSVKGRFTISRDNAKNTMSLQMNSLKPEDTAVYYCATPGIHGTSCNTSWKYYGQGTQVTVSS

SEQ ID NO: 19 - protein, 124 aa, synthetic construct
  REGION 1..124: note = sdAb
QVQLVESGGGLVQPGGSLRLSCVASGFTLDDYALGWFRQAPGKEREAVSCISGSDGGTHYHDSVKGRFTISKNKAKNTLYLQMNSLKPEDTAVYYCARAQADRCLGGIWPTGDWGQGTQVTVSS

SEQ ID NO: 20 - protein, 8 aa, synthetic construct
  REGION 1..8: note = CDR1
GFTFDDYA

SEQ ID NO: 21 - protein, 8 aa, synthetic construct
  REGION 1..8: note = CDR1
GFTLDYYA

SEQ ID NO: 22 - protein, 8 aa, synthetic construct
  REGION 1..8: note = CDR1
GLTLDGYA

SEQ ID NO: 23 - protein, 8 aa, synthetic construct
  REGION 1..8: note = CDR1
GFTLNEHA

SEQ ID NO: 24 - protein, 8 aa, synthetic construct
  REGION 1..8: note = CDR1
GSIESFSF

SEQ ID NO: 25 - protein, 8 aa, synthetic construct
  REGION 1..8: note = CDR1
GFFFDDYA

SEQ ID NO: 26 - protein, 8 aa, synthetic construct
  REGION 1..8: note = CDR1
GFTFDDTA

SEQ ID NO: 27 - protein, 8 aa, synthetic construct
  REGION 1..8: note = CDR1
GFTLDSYA

SEQ ID NO: 28 - protein, 8 aa, synthetic construct
  REGION 1..8: note = CDR1
GFTLDNYA

SEQ ID NO: 29 - protein, 8 aa, synthetic construct
  REGION 1..8: note = CDR1
GFIFDDYS

SEQ ID NO: 30 - protein, 8 aa, synthetic construct
  REGION 1..8: note = CDR1
GFTLDYYT

SEQ ID NO: 31 - protein, 8 aa, synthetic construct
  REGION 1..8: note = CDR1
GFTLDDYA

SEQ ID NO: 32 - protein, 8 aa, synthetic construct
  REGION 1..8: note = CDR2
IRSSDGST

SEQ ID NO: 33 - protein, 8 aa, synthetic construct
  REGION 1..8: note = CDR2
ISSSDGST

SEQ ID NO: 34 - protein, 8 aa, synthetic construct
  REGION 1..8: note = CDR2
ISGSDGST

SEQ ID NO: 35 - protein, 8 aa, synthetic construct
  REGION 1..8: note = CDR2
ISGTDGST

SEQ ID NO: 36 - protein, 10 aa, synthetic construct
  REGION 1..10: note = CDR2
FTGAASLSFS

SEQ ID NO: 37 - protein, 8 aa, synthetic construct
  REGION 1..8: note = CDR2
ISSSDGRI

SEQ ID NO: 38 - protein, 8 aa, synthetic construct
  REGION 1..8: note = CDR2
ITSSDGRT

SEQ ID NO: 39 - protein, 8 aa, synthetic construct
  REGION 1..8: note = CDR2
ISRNAGST

SEQ ID NO: 40 - protein, 8 aa, synthetic construct
  REGION 1..8: note = CDR2
ISSSDTKY

SEQ ID NO: 41 - protein, 8 aa, synthetic construct
  REGION 1..8: note = CDR2
ISGSDGGT

SEQ ID NO: 42 - protein, 14 aa, synthetic construct
  REGION 1..14: note = CDR3
AKDNYSDCRGPYHY

SEQ ID NO: 43 - protein, 18 aa, synthetic construct
  REGION 1..18: note = CDR3
ARDSLSGSGYGCRGPYDY

SEQ ID NO: 44 - protein, 13 aa, synthetic construct
  REGION 1..13: note = CDR3
AARRGSCSGSWTY

SEQ ID NO: 45 - protein, 17 aa, synthetic construct
  REGION 1..17: note = CDR3
AAALGRDCQGGQWKTSS

SEQ ID NO: 46 - protein, 13 aa, synthetic construct
  REGION 1..13: note = CDR3
NVDPWSQSWPGRY

SEQ ID NO: 47 - protein, 14 aa, synthetic construct
  REGION 1..14: note = CDR3
ATDRYSDCRGPYEN

SEQ ID NO: 48 - protein, 14 aa, synthetic construct
  REGION 1..14: note = CDR3
AADPYGDCRGPYDY

SEQ ID NO: 49 - protein, 14 aa, synthetic construct
  REGION 1..14: note = CDR3
AVRRGYGCSGPWHY

SEQ ID NO: 50 - protein, 17 aa, synthetic construct
  REGION 1..17: note = CDR3
AAALGRDCQGGQWKTDS

SEQ ID NO: 51 - protein, 17 aa, synthetic construct
  REGION 1..17: note = CDR3
AAARKGSTDCQGAIWGS

SEQ ID NO: 52 - protein, 14 aa, synthetic construct
  REGION 1..14: note = CDR3
AADRWQTCTGPYDH

SEQ ID NO: 53 - protein, 20 aa, synthetic construct
  REGION 1..20: note = CDR3
AARVPDYGDITDCGGKWDDY

SEQ ID NO: 54 - protein, 14 aa, synthetic construct
  REGION 1..14: note = CDR3
AARSGDGCNGRWDY

SEQ ID NO: 55 - protein, 16 aa, synthetic construct
  REGION 1..16: note = CDR3
ATPGIHGTSCNTSWKY

SEQ ID NO: 56 - protein, 17 aa, synthetic construct
  REGION 1..17: note = CDR3
ARAQADRCLGGIWPTGD

SEQ ID NO: 57 - protein, 5 aa, synthetic construct
  REGION 1..5: note = G4S linker
GGGGS

SEQ ID NO: 58 - protein, 288 aa, synthetic construct
  REGION 1..288: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSEPKSCDKTHTCPPCPAPELLG

SEQ ID NO: 59 - protein, 288 aa, synthetic construct
  REGION 1..288: note = sdAb fusion
MGWSCIILFLVATATGVHSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 60 - protein, 293 aa, synthetic construct
  REGION 1..293: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSEPKSCDKTHTCPPCPAPELLG

SEQ ID NO: 61 - protein, 293 aa, synthetic construct
  REGION 1..293: note = sdAb fusion
MGWSCIILFLVATATGVHSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 62 - protein, 298 aa, synthetic construct
  REGION 1..298: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSGGGGSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSEPKSCDKTHTCPPCPAPELLG

SEQ ID NO: 63 - protein, 298 aa, synthetic construct
  REGION 1..298: note = sdAb fusion
MGWSCIILFLVATATGVHSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSSGGGGSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 64 - protein, 266 aa, synthetic construct
  REGION 1..266: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSS

SEQ ID NO: 65 - protein, 287 aa, synthetic construct
  REGION 1..287: note = sdAb fusion
MGWSCIILFLVATATGVHSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 66 - protein, 292 aa, synthetic construct
  REGION 1..292: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSSEPKSCDKTHTCPPCPAPELLG

SEQ ID NO: 67 - protein, 292 aa, synthetic construct
  REGION 1..292: note = sdAb fusion
MGWSCIILFLVATATGVHSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 68 - protein, 297 aa, synthetic construct
  REGION 1..297: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSGGGGSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSSEPKSCDKTHTCPPCPAPELLG

SEQ ID NO: 69 - protein, 297 aa, synthetic construct
  REGION 1..297: note = sdAb fusion
MGWSCIILFLVATATGVHSQLQLVESGGGLVQPGGSLRLSCAASGLTLDGYAIGWFRQAPGKEREGLACISGSDGSTHYLDSVKGRFTISRDNGKNTMALQMNSLQFGDTAIYYCAARRGSCSGSWTYWGQGTQVTVSSGGGGSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 70 - protein, 291 aa, synthetic construct
  REGION 1..291: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSEPKSCDKTHTCPPCPAPELLG

SEQ ID NO: 71 - protein, 291 aa, synthetic construct
  REGION 1..291: note = sdAb fusion
MGWSCIILFLVATATGVHSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 72 - protein, 296 aa, synthetic construct
  REGION 1..296: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSEPKSCDKTHTCPPCPAPELLG

SEQ ID NO: 73 - protein, 296 aa, synthetic construct
  REGION 1..296: note = sdAb fusion
MGWSCIILFLVATATGVHSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 74 - protein, 301 aa, synthetic construct
  REGION 1..301: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSGGGGSGGGGSGGGGSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSEPKSCDKTHTCPPCPAPELLG

SEQ ID NO: 75 - protein, 301 aa, synthetic construct
  REGION 1..301: note = sdAb fusion
MGWSCIILFLVATATGVHSQLQLVESGGGLAQTGGSLRLSCAAAGFTFDDTATGWFRQAPGKNREGVACISSSDGRIHYIDSAKGRFTISKDNAKKTVYLQMDSLKPEDTAVYYCAAARKGSTDCQGAIWGSWGQGTQVIVSSGGGGSGGGGSGGGGSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLG

SEQ ID NO: 76 - protein, 499 aa, synthetic construct
  REGION 1..499: note = sdAb fusion
MGWSCIILFLVATATGVHSQLQLVESGGGLVQAGGSLRLSCAASGSIESFSFMGWYRQAPGKQRELVAGFTGAASLSFSTYTDAVKGRFTISRDNAKNTVYLQMNSLKPEDTAVYFCNVDPWSQSWPGRYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQVQLVESGGGLVQPGGSLRLSCAASGFTLDYYAIGWFRQAPGKEREGVSCIRSSDGSTYYADSVKGRFTISSDNTKNTVYLQMNSLKPEDTAVYYCATDRYSDCRGPYENWGQGTQVTVSS

SEQ ID NO: 77 - protein, 447 aa, synthetic construct
  REGION 1..447: note = VH of the BMS antibody
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 214 aa, synthetic construct
  REGION 1..214: note = VL of the BMS antibody
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 121 aa, synthetic construct
  REGION 1..121: note = GS2-2 antibody
QVQLVESGGGVVQPGGSLRLSCAASGLTLSQYTMGWFRQAPGKERELVAAIHWTSSVTDYADSVYGRFTISRDDSKNTGYLQMNSLRAEDTAVYYCAATHYYTHRGPFDYWGQGTLVTVSS

SEQ ID NO: 80 - protein, 120 aa, synthetic construct
  REGION 1..120: note = 17-H1 sdAb
QLQLVESGGGLVQPGGSLRLSCAASGLTLDAYAIGWFRQAPGKEREGLACISGSDGSTHYLDSVKGRFTISRDNGKNTMALQMNSLQFGDTAIYYCAARRGSCSGSWTYWGQGTQVTVSS

SEQ ID NO: 81 - protein, 120 aa, synthetic construct
  REGION 1..120: note = 17-H2 sdAb
QLQLVESGGGLVQPGGSLRLSCAASGLTLEGYAIGWFRQAPGKEREGLACISGSDGSTHYLDSVKGRFTISRDNGKNTMALQMNSLQFGDTAIYYCAARRGSCSGSWTYWGQGTQVTVSS

SEQ ID NO: 82 - protein, 120 aa, synthetic construct
  REGION 1..120: note = 17-H3 sdAb
QLQLVESGGGLVQPGGSLRLSCAASGLTLDGYAIGWFRQAPGKEREGLACISGSDASTHYLDSVKGRFTISRDNGKNTMALQMNSLQFGDTAIYYCAARRGSCSGSWTYWGQGTQVTVSS

SEQ ID NO: 83 - protein, 120 aa, synthetic construct
  REGION 1..120: note = 17-H4 sdAb
QLQLVESGGGLVQPGGSLRLSCAASGLTLDGYAIGWFRQAPGKEREGLACISGSEGSTHYLDSVKGRFTISRDNGKNTMALQMNSLQFGDTAIYYCAARRGSCSGSWTYWGQGTQVTVSS

SEQ ID NO: 84 - protein, 120 aa, synthetic construct
  REGION 1..120: note = 17-H5 sdAb
QLQLVESGGGLVQPGGSLRLSCAASGLTLDAYAIGWFRQAPGKEREGLACISGSDASTHYLDSVKGRFTISRDNGKNTMALQMNSLQFGDTAIYYCAARRGSCSGSWTYWGQGTQVTVSS

SEQ ID NO: 85 - protein, 121 aa, synthetic construct
  REGION 1..121: note = 27-H1
QVQLVESGGGLVQPGGSLRLSCAASGFTLDYYAIGWFRQAPGKEREGVSCIRSSDASTYYADSVKGRFTISSDNTKNTVYLQMNSLKPEDTAVYYCATDRYSDCRGPYENWGQGTQVTVSS

SEQ ID NO: 86 - protein, 121 aa, synthetic construct
  REGION 1..121: note = 27-H2 sdAb
QVQLVESGGGLVQPGGSLRLSCAASGFTLDYYAIGWFRQAPGKEREGVSCIRSSEGSTYYADSVKGRFTISSDNTKNTVYLQMNSLKPEDTAVYYCATDRYSDCRGPYENWGQGTQVTVSS

SEQ ID NO: 87 - protein, 448 aa, synthetic construct
  REGION 1..448: note = KN035 HC
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 214 aa, synthetic construct
  REGION 1..214: note = KN035 LC
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 120 aa, synthetic construct
  REGION 1..120: note = 17-H5B1 sdAb
EVQLVESGGGLVQPGGSLRLSCAASGLTLDAYAIGWFRQAPGKEREGVACISGSDASTHYLDSVKGRFTISRDNSKNTLYLQMNSLRAEDTAVYYCAARRGSCSGSWTYWGQGTTVTVSS

SEQ ID NO: 90 - protein, 120 aa, synthetic construct
  REGION 1..120: note = 17-H5B2 sdAb
QVQLVESGGGLVQPGGSLRLSCAASGLTLDAYAIGWFRQAPGKEREGVACISGSDASTHYLDSVKGRFTISRDNSKNTMYLQMNSLRAEDTAVYYCAARRGSCSGSWTYWGQGTTVTVSS

SEQ ID NO: 91 - protein, 120 aa, synthetic construct
  REGION 1..120: note = 17-H5B3 sdAb
QLQLVESGGGLVQPGGSLRLSCAASGLTLDAYAIGWFRQAPGKEREGLACISGSDASTHYLDSVKGRFTISRDNSKNTMYLQMNSLRAEDTAVYYCAARRGSCSGSWTYWGQGTTVTVSS

SEQ ID NO: 92 - protein, 122 aa, synthetic construct
  REGION 1..122: note = 25-B5 sdAb
QVQLVESGGGLVKPGGSLRLSCAASGSIESFSFMGWYRQAPGKQREWVAGFTGAASLSFSTYTDAVKGRFTISRDNAKNSLYLQMNSLRAEDTAVYYCNVDPWSQSWPGRYWGQGTTVTVSS

SEQ ID NO: 93 - protein, 122 aa, synthetic construct
  REGION 1..122: note = 25-B6 sdAb
QLQLVESGGGLVKPGGSLRLSCAASGSIESFSFMGWYRQAPGKQREWVAGFTGAASLSFSTYTDAVKGRFTISRDNAKNSVYLQMNSLRAEDTAVYYCNVDPWSQSWPGRYWGQGTTVTVSS

SEQ ID NO: 94 - protein, 122 aa, synthetic construct
  REGION 1..122: note = 25-B7 sdAb
QLQLVESGGGLVKPGGSLRLSCAASGSIESFSFMGWYRQAPGKQRELVAGFTGAASLSFSTYTDAVKGRFTISRDNAKNSVYLQMNSLRAEDTAVYFCNVDPWSQSWPGRYWGQGTTVTVSS

SEQ ID NO: 95 - protein, 121 aa, synthetic construct
  REGION 1..121: note = 27-H1B1 sdAb
EVQLVESGGGLVQPGGSLRLSCAASGFTLDYYAIGWFRQAPGKEREWVSCIRSSDASTYYADSVKGRFTISSDNSKNTLYLQMNSLRAEDTAVYYCATDRYSDCRGPYENWGQGTMVTVSS

SEQ ID NO: 96 - protein, 121 aa, synthetic construct
  REGION 1..121: note = 27-H1B2
QVQLVESGGGLVQPGGSLRLSCAASGFTLDYYAIGWFRQAPGKEREWVSCIRSSDASTYYADSVKGRFTISSDNSKNTLYLQMNSLRAEDTAVYYCATDRYSDCRGPYENWGQGTMVTVSS

SEQ ID NO: 97 - protein, 121 aa, synthetic construct
  REGION 1..121: note = 27-H1B3 sdAb
QVQLVESGGGLVQPGGSLRLSCAASGFTLDYYAIGWFRQAPGKEREGVSCIRSSDASTYYADSVKGRFTISSDNSKNTVYLQMNSLRAEDTAVYYCATDRYSDCRGPYENWGQGTMVTVSS

SEQ ID NO: 98 - protein, 10 aa, synthetic construct
  REGION 1..10: note = CDR1
GLTLDGYAIG

SEQ ID NO: 99 - protein, 10 aa, synthetic construct
  REGION 1..10: note = CDR1
GLTLDAYAIG

SEQ ID NO: 100 - protein, 10 aa, synthetic construct
  REGION 1..10: note = CDR1
GLTLEGYAIG

SEQ ID NO: 101 - protein, 10 aa, synthetic construct
  REGION 1..10: note = CDR1
GFTLDYYAIG

SEQ ID NO: 102 - protein, 17 aa, synthetic construct
  REGION 1..17: note = CDR2
CISGSDGSTHYLDSVKG

SEQ ID NO: 103 - protein, 17 aa, synthetic construct
  REGION 1..17: note = CDR2
CISGSDASTHYLDSVKG

SEQ ID NO: 104 - protein, 17 aa, synthetic construct
  REGION 1..17: note = CDR2
CISGSEGSTHYLDSVKG

SEQ ID NO: 105 - protein, 17 aa, synthetic construct
  REGION 1..17: note = CDR2
CIRSSDGSTYYADSVKG

SEQ ID NO: 106 - protein, 17 aa, synthetic construct
  REGION 1..17: note = CDR2
CIRSSDASTYYADSVKG

SEQ ID NO: 107 - protein, 17 aa, synthetic construct
  REGION 1..17: note = CDR2
CIRSSEGSTYYADSVKG

SEQ ID NO: 108 - protein, 11 aa, synthetic construct
  REGION 1..11: note = CDR3
RRGSCSGSWTY

SEQ ID NO: 109 - protein, 12 aa, synthetic construct
  REGION 1..12: note = CDR3
DRYSDCRGPYEN

SEQ ID NO: 110 - protein, 10 aa, synthetic construct
  REGION 1..10: note = CDR1
GSIESFSFMG

SEQ ID NO: 111 - protein, 19 aa, synthetic construct
  REGION 1..19: note = CDR2
GFTGAASLSFSTYTDAVKG

SEQ ID NO: 112 - protein, 11 aa, synthetic construct
  REGION 1..11: note = CDR3
DPWSQSWPGRY